SEQUENCE LISTING (27 sequences)

SEQ ID NO: 1 - DNA, 270 bp, Homo sapiens
  source 1..270: mol_type = genomic DNA
  misc_feature 126^127: note = cryptic splice site
ggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccag

SEQ ID NO: 2 - DNA, 270 bp, Homo sapiens
  source 1..270: mol_type = genomic DNA
  misc_feature 126^127: note = cryptic splice site
ggctcccactgcagcagctcgggggaccccgctgagtacaacctgcgctcgcgcaccgtgctgtgcgggacctgcgggcagcctgccgacaaggcatctgccagcggctcaggagcccaggtgggtggacccatctcctctggctcttctgcctccagtgtcacggtcactcgcagctaccgcagtgtggggggcagtgggggtggcagcttcggggacaatctggtcacccgctcctacctcctgggcaactccagcccccgaacccag

SEQ ID NO: 3 - DNA, 25 bp, synthetic construct
  misc_feature 1..25: note = antisense oligonucleotide
  source 1..25: mol_type = other DNA
ctgagccgctggcagatgccttgtc

SEQ ID NO: 4 - DNA, 25 bp, synthetic construct
  misc_feature 1..25: note = antisense oligonucleotide
  source 1..25: mol_type = other DNA
gaggagatgggtccacccacctggg

SEQ ID NO: 5 - protein, 9 aa, synthetic construct
  REGION 1..9: note = cell penetrating peptide
RRRQRRKKR

SEQ ID NO: 6 - protein, 9 aa, synthetic construct
  REGION 1..9: note = cell penetrating peptide
RKKRRQRRR

SEQ ID NO: 7 - protein, 11 aa, synthetic construct
  REGION 1..11: note = cell penetrating peptide
RRRRRRRRRFF

SEQ ID NO: 8 - protein, 11 aa, synthetic construct
  REGION 1..11: note = cell penetrating peptide
RRRRRFFRRRR

SEQ ID NO: 9 - protein, 4 aa, synthetic construct
  REGION 1..4: note = cell penetrating peptide
RRRR

SEQ ID NO: 10 - protein, 5 aa, synthetic construct
  REGION 1..5: note = cell penetrating peptide
RRRRR

SEQ ID NO: 11 - protein, 6 aa, synthetic construct
  REGION 1..6: note = cell penetrating peptide
RRRRRR

SEQ ID NO: 12 - protein, 7 aa, synthetic construct
  REGION 1..7: note = cell penetrating peptide
RRRRRRR

SEQ ID NO: 13 - protein, 8 aa, synthetic construct
  REGION 1..8: note = cell penetrating peptide
RRRRRRRR

SEQ ID NO: 14 - protein, 9 aa, synthetic construct
  REGION 1..9: note = cell penetrating peptide
RRRRRRRRR

SEQ ID NO: 15 - protein, 16 aa, synthetic construct
  REGION 1..16: note = cell penetrating peptide
  MOD_RES 2: note = X= 6-aminohexanoic acid
  MOD_RES 4: note = X= 6-aminohexanoic acid
  MOD_RES 6: note = X= 6-aminohexanoic acid
  MOD_RES 8: note = X= 6-aminohexanoic acid
  MOD_RES 10: note = X= 6-aminohexanoic acid
  MOD_RES 12: note = X= 6-aminohexanoic acid
  MOD_RES 14: note = X= 6-aminohexanoic acid
  MOD_RES 16: note = X= 6-aminohexanoic acid
RXRXRXRXRXRXRXRX

SEQ ID NO: 16 - protein, 12 aa, synthetic construct
  REGION 1..12: note = cell penetrating peptide
  MOD_RES 2: note = X= 6-aminohexanoic acid
  MOD_RES 5: note = X= 6-aminohexanoic acid
  MOD_RES 8: note = X= 6-aminohexanoic acid
  MOD_RES 11: note = X= 6-aminohexanoic acid
RXRRXRRXRRXR

SEQ ID NO: 17 - protein, 15 aa, synthetic construct
  REGION 1..15: note = cell penetrating peptide
  MOD_RES 2: note = X= 6-aminohexanoic acid
  MOD_RES 5: note = X= 6-aminohexanoic acid
  MOD_RES 8: note = X= 6-aminohexanoic acid
  MOD_RES 11: note = X= 6-aminohexanoic acid
  MOD_RES 14: note = X= 6-aminohexanoic acid
RXRRXRRXRRXRRXR

SEQ ID NO: 18 - protein, 12 aa, synthetic construct
  REGION 1..12: note = cell penetrating peptide
  MOD_RES 2: note = X= 6-aminohexanoic acid
  MOD_RES 5: note = X = beta-alanine
  MOD_RES 8: note = X= 6-aminohexanoic acid
  MOD_RES 11: note = X = beta-alanine
RXRRXRRXRRXR

SEQ ID NO: 19 - protein, 14 aa, synthetic construct
  REGION 1..14: note = cell penetrating peptide
RARRARRARRARFF

SEQ ID NO: 20 - protein, 14 aa, synthetic construct
  REGION 1..14: note = cell penetrating peptide
RGRRGRRGRRGRFF

SEQ ID NO: 21 - protein, 10 aa, synthetic construct
  REGION 1..10: note = cell penetrating peptide
RFFRFFRFFR

SEQ ID NO: 22 - protein, 14 aa, synthetic construct
  REGION 1..14: note = cell penetrating peptide
  MOD_RES 14: note = X = beta-alanine
  MOD_RES 2: note = X = 6-aminohexanoic acid
  MOD_RES 5: note = X = 6-aminohexanoic acid
  MOD_RES 8: note = X = 6-aminohexanoic acid
  MOD_RES 11: note = X = 6-aminohexanoic acid
  MOD_RES 13: note = X = 6-aminohexanoic acid
RXRRXRRXRRXRXX

SEQ ID NO: 23 - protein, 12 aa, synthetic construct
  REGION 1..12: note = cell penetrating peptide
  MOD_RES 11: note = X = 6-aminohexanoic acid
  MOD_RES 12: note = X = beta-alanine
RFFRFFRFFRXX

SEQ ID NO: 24 - protein, 11 aa, synthetic construct
  REGION 1..11: note = cell penetrating peptide
RFFRFFRFFRG

SEQ ID NO: 25 - protein, 7 aa, synthetic construct
  REGION 1..7: note = cell penetrating peptide
RRRRRRG

SEQ ID NO: 26 - protein, 15 aa, synthetic construct
  MOD_RES 2: note = X = beta-alanine
  MOD_RES 5: note = X = beta-alanine
  MOD_RES 12: note = X = beta-alanine
  MOD_RES 14: note = X = beta-alanine
  REGION 1..15: note = cell penetrating peptide
RXRRXRFQILYXRXR

SEQ ID NO: 27 - protein, 16 aa, synthetic construct
  REGION 1..16: note = cell penetrating peptide
  MOD_RES 2: note = X = beta-alanine
  MOD_RES 5: note = X = beta-alanine
  MOD_RES 13: note = X = beta-alanine
  MOD_RES 15: note = X = beta-alanine
RXRRXRFQILYRXHXH

(19) United States
(12) Patent Application Publication
(10) Pub. No.: US 2024/0218365 A1
(43) Pub. Date: Jul. 4, 2024

(54) FORMULATION OF AN ANTISENSE OLIGOMER CONJUGATE

(71) Applicant: SAREPTA THERAPEUTICS, INC., Cambridge, MA (US)

(72) Inventors: Leslie GORDON, Foxboro, MA (US); Raymond Skwierczynski, Peabody, MA (US); Adam CHOI, Woburn, MA (US); Luca Ogunleye, Woburn, MA (US)

(21) Appl. No.: 18/500,627

(22) Filed: Nov. 2, 2023

Related U.S. Application Data
(60) Provisional application No. 63/382,043, filed on Nov. 2, 2022.

(51) Int. Cl. C12N 15/113; A61K 47/10; A61K 47/18; A61K 47/26; A61K 47/62

(52) U.S. Cl. CPC C12N 15/113; A61K 47/10; A61K 47/183; A61K 47/26; A61K 47/62; C12N 2310/11; C12N 2320/33

(57) ABSTRACT

Provided herein are pharmaceutical compositions comprising benzyl alcohol and an antisense oligomer conjugate of formula (1):

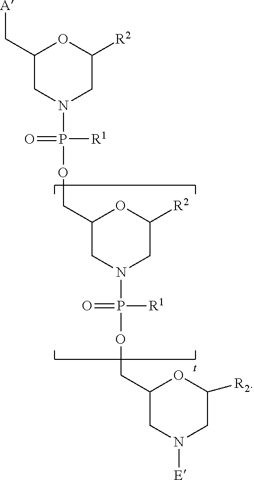

Also provided herein are methods of treating progeroid diseases, such as Hutchinson-Gilford progeria syndrome (HGPS), in a subject in need thereof, comprising administering to the subject a pharmaceutical composition as described herein.

### CROSS-REFERENCE TO RELATED APPLICATIONS

This application claims the benefit of U.S. Provisional Patent Application No. 63/382,043, filed Nov. 2, 2022, the entire content of which is incorporated herein by reference.

### STATEMENT REGARDING SEQUENCE LISTING

The Sequence Listing associated with this application is provided in xml format in lieu of a paper copy, and is hereby incorporated by reference into the specification. The name of the text file containing the Sequence Listing is 720158-SPT-8192_SEQ_LISTING.xml. The text file is about 56 KB, was created on Jan. 11, 2024, and is being submitted electronically via EFS web.

### BACKGROUND

Antisense technology provides a means for modulating the expression of one or more specific gene products, including alternative splice products, and is uniquely useful in a number of therapeutic, diagnostic, and research applications. The principle behind antisense technology is that an antisense compound, e.g., an oligonucleotide, which hybridizes to a target nucleic acid, modulates gene expression activities such as transcription, splicing or translation through any one of a number of antisense mechanisms. The sequence specificity of antisense compounds makes them attractive as tools for target validation and gene functionalization, as well as therapeutics to selectively modulate the expression of genes involved in disease.

Although significant progress has been made in the field of antisense technology, there remains a need in the art for oligonucleotides and peptide-oligonucleotide-conjugates with improved antisense or antigene performance. Such improved antisense or antigene performance includes, at least, for example: lower toxicity, stronger affinity for DNA and RNA without compromising sequence selectivity, improved pharmacokinetics and tissue distribution, improved cellular delivery, and both reliable and controllable in vivo distribution.

Hutchinson-Gilford progeria syndrome (HGPS) is a rare genetic disorder characterized by premature arteriosclerosis and degeneration of vascular smooth muscle cells (SMCs). HGPS manifests itself most notably as accelerated, premature aging in affected children. Children with HGPS have progressive symptoms such as growth retardation, alopecia, loss of subcutaneous fat, and bone abnormalities. Average lifespan is 12 years with the most common cause of death being myocardial infarction or stroke.

Most HGPS cases are caused by a single-point mutation in the lamin A (LMNA) gene, resulting in the generation of progerin, a truncated splicing mutant of lamin A. The single-point mutation is a de novo silent substitution (1824C>T, Gly608Gly) in exon 11 of the lamin A (LMNA) gene. The substitution activates a cryptic splice donor site, which leads to the production of a dominant negative mutant lamin A protein with an internal deletion of 50 amino acids. The mutant protein, named progerin, accumulates on the nuclear membrane, causing characteristic nuclear blebbing ((Scaffidi and Misteli 2005; Cao, Blair et al. 2011)).

It is known that aberrant splicing can be corrected using phosphorodiamidate morpholino oligonucleotides (PMOs), or more specifically, splice-switching oligonucleotides (SSOs). SSOs block aberrant splicing sites by hybridizing at or near the sites thereby preventing recognition by the cellular splicing machinery. Exemplary SSOs are resistant to nucleases and the resulting double-stranded structure eliminates the possibility of RNA cleavage by RNase H. SSOs have been shown to effectively repair the splicing pattern both in vitro and in vivo for thalassemia and Duchenne muscular dystrophy. (Kinali, Arechavala-Gomeza et al. 2009; Svasti, Suwanmanee et al. 2009). The aberrant splicing of LMNA associated with HGPS has been shown to be reduced by correction of the aberrant splicing event using modified antisense oligonucleotides targeted to the activated cryptic splice site both in cell culture (Scaffidi and Misteli 2005) and in a relevant animal model (Osorio, Navarro et al. 2011).

Given the role of LMNA in HGPS, oligonucleotides that modulate splicing of LMNA pre-mRNA to eliminate expression of progerin are needed.

### SUMMARY

The present disclosure provides, inter alia, a pharmaceutical composition comprising benzyl alcohol and an antisense oligomer conjugate of formula (I):

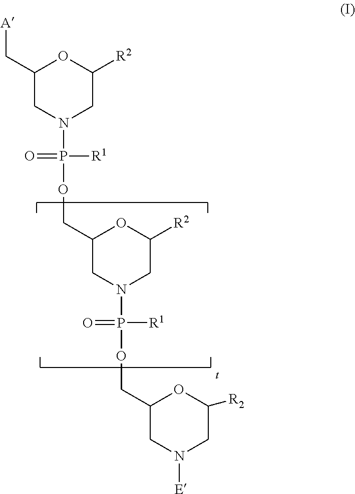

or a pharmaceutically acceptable salt thereof, wherein A′ R¹, R², t, and E′ are as defined herein. The present disclosure also provides a method for treating Hutchinson-Gilford progeria syndrome (HGPS) in a subject in need thereof comprising administering to the subject a pharmaceutical composition of the present disclosure.

### BRIEF DESCRIPTION OF THE DRAWINGS

FIG. 1 shows the effect of the concentration of an antisense oligomer conjugate of the present disclosure on viscosity.

FIG. 2 shows an SCX-HPLC overlay of antisense oligomer conjugate test solutions at various concentrations. The monomeric species and higher-order species, resulting from aggregation, are identified.

FIG. 3 shows aggregation profiles for compositions of an antisense oligomer conjugate of the disclosure and an additional excipient in citrate buffer. For each excipient, data for to is presented in the left bar and for t_{7day} in the right bar.

FIG. 4 shows aggregation profiles for compositions of an antisense oligomer conjugate of the disclosure and an excipient in phosphate buffer. For each excipient, data for to is presented in the left bar and for t_{7day} in the right bar.

FIG. 5 shows aggregation profiles for compositions of an antisense oligomer conjugate of the disclosure and an excipient in either citrate or phosphate buffer after seven days. For each excipient, data for the citrate-buffered composition is presented in the left bar and for the phosphate-buffered composition in the right bar.

FIG. 6 shows aggregation profiles for compositions of an antisense oligomer conjugate of the disclosure. For each composition, data for to (rt) is presented in the left bar, data for t_{7day} (rt) in the middle bar, and data for t_{7day} (2-8° C.) in the right bar.

FIG. 7 shows aggregation profiles for compositions of an antisense oligomer conjugate of the disclosure. For each composition, data for to (rt) is presented as the left-most bar, data for t_{7day} (rt) in the central-left bar, data for t_{7day} (2-8° C.) in central-right bar, and data for t_{14day} (rt) in the right-most bar.

FIG. 8 shows aggregation profiles for compositions of an antisense oligomer conjugate of the disclosure held at 25° C. For each composition, data for to is presented in the left bar, data for t_{7day} in the middle bar, and data for t_{14day} in the right bar.

FIG. 9 shows aggregation profiles for compositions of an antisense oligomer conjugate of the disclosure held at 60° C. For each composition, data for to is presented in the left bar, data for t_{7day} in the middle bar, and data for t_{14day} in the right bar.

FIG. 10 shows a degradation rate constant plot assuming first-order kinetics.

FIG. 11 shows a degradation rate constant plot assuming second-order kinetics.

FIG. 12 shows a set of Arrhenius plots used in calculating the estimated shelf life of a pharmaceutical composition of the present disclosure.

FIG. 13 shows the calculation of the shelf life of a pharmaceutical composition of the present disclosure.

### DETAILED DESCRIPTION

The present disclosure relates to pharmaceutical compositions comprising benzyl alcohol and an antisense oligomer conjugate of formula (I), as defined herein. The conjugates of the present disclosure are susceptible to the formation of higher order species (i.e., aggregates) upon storage under various conditions. Applicant has surprisingly found that a composition comprising a conjugate of the present disclosure and benzyl alcohol is resistant to aggregation and is significantly and unexpectedly more stable than a composition lacking benzyl alcohol. Indeed, the pharmaceutical compositions of the present disclosure have been shown to be highly stable; compatible with both PES and PVDF membranes; resistant to degradation under photolytic, shear, and freeze-thaw stress; and are predicted to have a shelf life at 2-8° C. for at least five-years.

### Definitions

Listed below are definitions of various terms used to describe this disclosure. These definitions apply to the terms as they are used throughout this specification and claims, unless otherwise limited in specific instances, either individually or as part of a larger group.

The term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂—CH₂—S(═O)—CH₃. Up to two heteroatoms may be consecutive, such as, for example, —CH₂—NH—OCH₃, or —CH₂—CH₂—S—S—CH₃.

The term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. In various embodiments, examples of an aryl group may include phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆₋₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁₋₉-heteroaryl indicates the number of carbon atoms contained in the heteroaryl group without including the number of heteroatoms. For example, a C₁₋₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

The term “protecting group” or “chemical protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, monomethoxytrityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid moities may be blocked with base labile groups such as, without limitation, methyl, or ethyl, and hydroxy reactive moieties may be blocked with base labile groups such as acetyl in the presence of amines blocked with acid labile groups such as tert-butyl carbamate or with carbamates that are both acid and base stable but hydrolytically removable.

Carboxylic acid and hydroxyl reactive moieties may also be blocked with hydrolytically removable protective groups such as the benzyl group, while amine groups may be blocked with base labile groups such as Fmoc. A particularly useful amine protecting group for the synthesis of compounds of Formula (I) is the trifluoroacetamide. Carboxylic acid reactive moieties may be blocked with oxidatively-removable protective groups such as 2,4-dimethoxybenzyl, while coexisting amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

The term “nucleobase,” “base pairing moiety,” “nucleobase-pairing moiety,” or “base” refers to the heterocyclic ring portion of a nucleoside, nucleotide, and/or morpholino subunit. Nucleobases may be naturally occurring, or may be modified or analogs of these naturally occurring nucleobases, e.g., one or more nitrogen atoms of the nucleobase may be independently at each occurrence replaced by carbon. Exemplary analogs include hypoxanthine (the base component of the nucleoside inosine); 2, 6-diaminopurine; 5-methyl cytosine; C5-propynyl-modified pyrimidines; 10-(9-(aminoethoxy)phenoxazinyl) (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313, are also contemplated, the contents of which are incorporated herein by reference.

Further examples of base pairing moieties include, but are not limited to, expanded-size nucleobases in which one or more benzene rings have been added. Nucleic base replacements described in the Glen Research catalog (www.glenresearch.com); Krueger A T et al., Acc. Chem. Res., 2007, 40, 141-150; Kool, ET, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, the contents of which are incorporated herein by reference, are contemplated as useful for the synthesis of the oligomers described herein. Examples of expanded-size nucleobases are shown below:

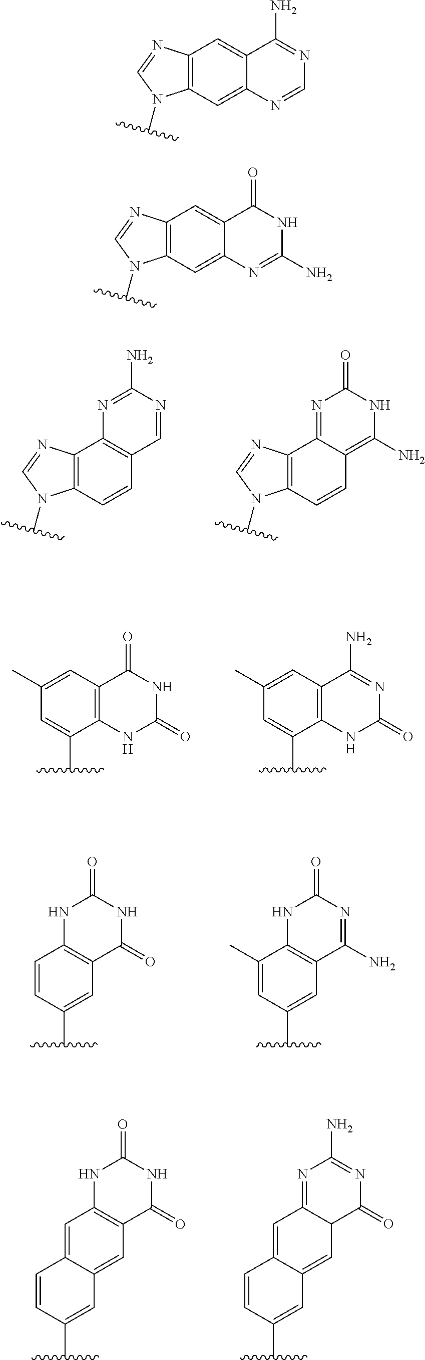

The terms “oligonucleotide” or “oligomer” refer to a compound comprising a plurality of linked nucleosides, nucleotides, or a combination of both nucleosides and nucleotides. In specific embodiments provided herein, an oligonucleotide is a morpholino oligonucleotide.

The phrase “morpholino oligonucleotide” or “PMO” refers to a modified oligonucleotide having morpholino subunits linked together by phosphoramidate or phosphorodiamidate linkages, joining the morpholino nitrogen of one subunit to the 5′-exocyclic carbon of an adjacent subunit. Each morpholino subunit comprises a nucleobase-pairing moiety effective to bind, by nucleobase-specific hydrogen bonding, to a nucleobase in a target.

The terms “antisense oligomer,” “antisense compound” and “antisense oligonucleotide” are used interchangeably and refer to a sequence of subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. The oligomer may have exact (perfect) or near (sufficient) sequence complementarity to the target sequence; variations in sequence near the termini of an oligomer are generally preferable to variations in the interior.

Such an antisense oligomer can be designed to block or inhibit translation of mRNA or to inhibit/alter natural or abnormal pre-mRNA splice processing, and may be said to be “directed to” or “targeted against” a target sequence with which it hybridizes. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a preprocessed mRNA. In various embodiments, a target sequence may be any region of a preprocessed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

The antisense oligonucleotide and the target RNA are complementary to each other when a sufficient number of corresponding positions in each molecule are occupied by nucleotides which can hydrogen bond with each other, such that stable and specific binding occurs between the oligonucleotide and the target. Thus, “specifically hybridizable” and “complementary” are terms which are used to indicate a sufficient degree of complementarity or precise pairing such that stable and specific binding occurs between the oligonucleotide and the target. It is understood in the art that the sequence of an oligonucleotide need not be 100% complementary to that of its target sequence to be specifically hybridizable. An oligonucleotide is specifically hybridizable when binding of the oligonucleotide to the target molecule interferes with the normal function of the target RNA, and there is a sufficient degree of complementarity to avoid non-specific binding of the antisense oligonucleotide to non-target sequences under conditions in which specific binding is desired, i.e., under physiological conditions in the case of in vivo assays or therapeutic treatment, and in the case of in vitro assays, under conditions in which the assays are performed.

Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases. In some embodiments, the nucleobase is covalently linked at the N9 atom of the purine base, or at the N1 atom of the pyrimidine base, to the morpholine ring of a nucleotide or nucleoside.

Purine bases comprise a pyrimidine ring fused to an imidazole ring, as described by the general formula:

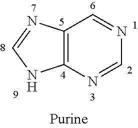

Adenine and guanine are the two purine nucleobases most commonly found in nucleic acids. These may be substituted with other naturally-occurring purines, including but not limited to N6-methyladenine, N2-methylguanine, hypoxanthine, and 7-methylguanine.

Pyrimidine bases comprise a six-membered pyrimidine ring as described by the general formula:

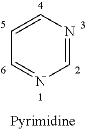

Cytosine, uracil, and thymine are the pyrimidine bases most commonly found in nucleic acids. These may be substituted with other naturally-occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Pseudouracil is a naturally occurring isomerized version of uracil, with a C-glycoside rather than the regular N-glycoside as in uridine.

Certain modified or substituted nucleobases are particularly useful for increasing the binding affinity of the antisense oligonucleotides of the disclosure. These include 5-substituted pyrimidines, 6-azapyrimidines and N-2, N-6 and 0-6 substituted purines, including 2-aminopropyladenine, 5-propynyluracil and 5-propynylcytosine. In various embodiments, nucleobases may include 5-methylcytosine substitutions, which have been shown to increase nucleic acid duplex stability by 0.6-1.2° ° C.

In some embodiments, modified or substituted nucleobases are useful for facilitating purification of antisense oligonucleotides. For example, in certain embodiments, antisense oligonucleotides may contain three or more (e.g., 3, 4, 5, 6 or more) consecutive guanine bases. In certain antisense oligonucleotides, a string of three or more consecutive guanine bases can result in aggregation of the oligonucleotides, complicating purification. In such antisense oligonucleotides, one or more of the consecutive guanines can be substituted with hypoxanthine. The substitution of hypoxanthine for one or more guanines in a string of three or more consecutive guanine bases can reduce aggregation of the antisense oligonucleotide, thereby facilitating purification.

The oligonucleotides provided herein are synthesized and do not include antisense compositions of biological origin. The molecules of the disclosure may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution, or absorption, or a combination thereof.

The terms “complementary” and “complementarity” refer to oligonucleotides (i.e., a sequence of nucleotides) related by base-pairing rules. For example, the sequence “T-G-A (5′-3′),” is complementary to the sequence “T-C-A (5′-3′).” Complementarity may be “partial,” in which only some of the nucleic acids' bases are matched according to base pairing rules. Or, there may be “complete,” “total,” or “perfect” (100%) complementarity between the nucleic acids. The degree of complementarity between nucleic acid strands has significant effects on the efficiency and strength of hybridization between nucleic acid strands. While perfect complementarity is often desired, some embodiments can include one or more but preferably 6, 5, 4, 3, 2, or 1 mismatches with respect to the target RNA. Such hybridization may occur with “near” or “substantial” complementarity of the antisense oligomer to the target sequence, as well as with exact complementarity. In some embodiments, an oligomer may hybridize to a target sequence at about 50%, 55%, 60%, 65%, 70%, 75%, 80%, 85%, 90%, 95%, 99% or 100% complementarity. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′-terminus, 3′-terminus, or both termini.

The term “naturally occurring amino acid” refers to an amino acid present in proteins found in nature, such as the 20 (L)-amino acids utilized during protein biosynthesis as well as others such as 4-hydroxyproline, hydroxylysine, desmosine, isodesmosine, homocysteine, citrulline and ornithine. The term “non-natural amino acids” refers to those amino acids not present in proteins found in nature, examples include beta-alanine (β-Ala), 6-aminohexanoic acid (Ahx) and 6-aminopentanoic acid. Additional examples of “non-natural amino acids” include, without limitation, (D)-amino acids, norleucine, norvaline, p-fluorophenylalanine, ethionine and the like, which are known to a person skilled in the art.

The term “peptide” refers to a compound comprising a plurality of linked amino acids. The peptides provided herein can be considered to be cell penetrating peptides.

The terms “cell penetrating peptide” and “CPP” are used interchangeably and refer to cationic cell penetrating peptides, also called transport peptides, carrier peptides, or peptide transduction domains. The peptides, provided herein, have the capability of inducing cell penetration within 100% of cells of a given cell culture population and allow macromolecular translocation within multiple tissues in vivo upon systemic administration. In various embodiments, a CPP embodiment of the disclosure may include an arginine-rich peptide as described further below.

As used herein, the term “treatment” or “treating,” is defined as the application or administration of a therapeutic agent, i.e., a conjugate of the present disclosure (in the form of a pharmaceutical composition), to a patient, or application or administration of a therapeutic agent to an isolated tissue or cell line from a patient (e.g., for diagnosis or ex vivo applications). Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

An “effective amount” or “therapeutically effective amount” refers to an amount of therapeutic compound, such as an antisense oligomer, administered to a mammalian subject, either as a single dose or as part of a series of doses, which is effective to produce a desired therapeutic effect.

The term “amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

As used herein, “pharmaceutically acceptable salts” refers to derivatives of the disclosed oligonucleotides wherein the parent oligonucleotide is modified by converting an existing acid or base moiety to its salt form. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Pharmaceutical Compositions

Provided herein are pharmaceutical compositions comprising benzyl alcohol and an antisense oligomer conjugate of formula (I):

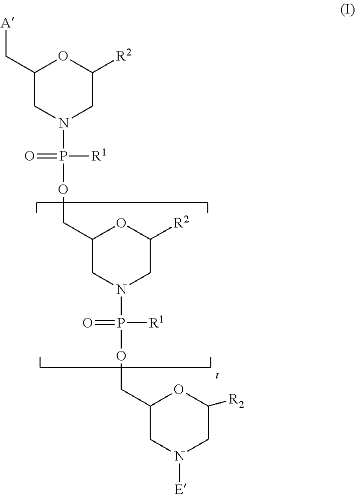

or a pharmaceutically acceptable salt thereof,
wherein:
A′ is selected from —OH,

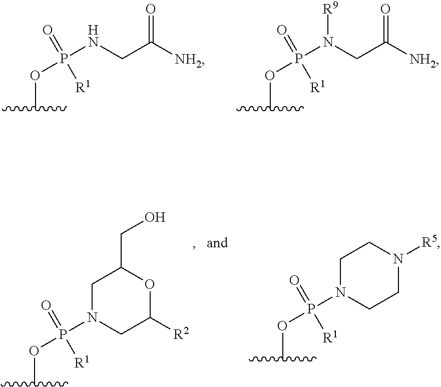

wherein
R⁵ is —C(O)(O-alkyl)ₓ—OH, wherein x is 3-10 and each alkyl group is, independently at each occurrence, C₂₋₆-alkyl,
or R⁵ is selected from —H, —C(O)C₁₋₆-alkyl, trityl, monomethoxytrityl, —(C₁₋₆-alkyl)-R⁶, —(C₁₋₆-heteroalkyl)-R⁶, —C₆₋₁₀-aryl-R⁶, 5- to 10-membered heteroaryl-R⁶, —C(O)O—(C₁₋₆-alkyl)-R⁶, —C(O)O—(C₆₋₁₀-aryl)-R⁶, —C(O)O-(5- to 10-membered heteroaryl)-R⁶, and

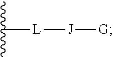

R⁶ is selected from —OH, —SH, and —NH₂, or R⁶ is O, S, or NH, each of which is covalently linked to a solid support;
R⁹ is C₁₋₆-alkyl;
each R′ is independently selected from —OH and —N(R³)(R⁴), wherein each R³ and R⁴ is, independently at each occurrence, —H or —C₁₋₆-alkyl;
each R² is independently, at each occurrence, selected from —H, a nucleobase, and a nucleobase functionalized with a chemical protecting group, wherein the nucleobase and the nucleobase functionalized with a chemical protecting group, independently at each occurrence, comprise a ring selected from pyridine, pyrimidine, purine, and deaza-purine;
t is 8-40;
E′ is selected from —H, —C₁₋₆-alkyl, —C(O)C₁₋₆-alkyl, benzoyl, stearoyl, trityl, monomethoxytrityl, dimethoxytrityl, trimethoxytrityl,

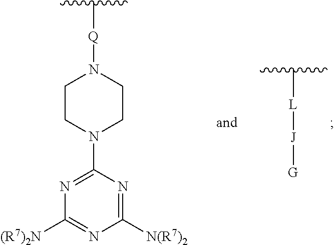

wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)(NHC(═NH)NH₂;
L is a linking amino acid, wherein L is covalently linked by an amide bond to the N-terminus or C-terminus of J;
J is a cell-penetrating peptide;
G is selected from —H, —C(O)C₁₋₆-alkyl, benzoyl, and stearoyl, wherein G is covalently linked to J; and
wherein at least one of the following is true:
(1) A′ is

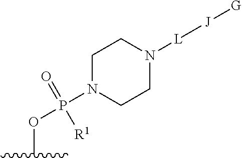

or (2) E′ is

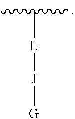

### Antisense Oligomer Conjugates of Formula (I)

The pharmaceutical compositions of the present disclosure comprise an antisense oligomer conjugate of formula (I), as defined herein. Certain embodiments of the antisense oligomer conjugates of formula (I) are as follows.

In some embodiments of the conjugate of formula (I), A′ is selected from —OH,

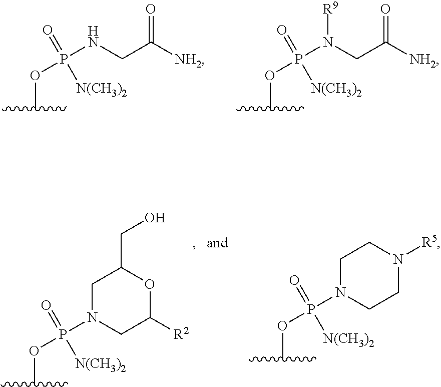

In some embodiments of the conjugate of formula (I), A′ is selected from

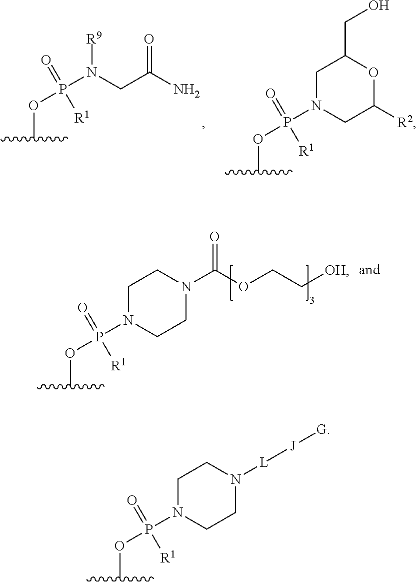

In some embodiments of the conjugate of formula (I), A′ is selected from

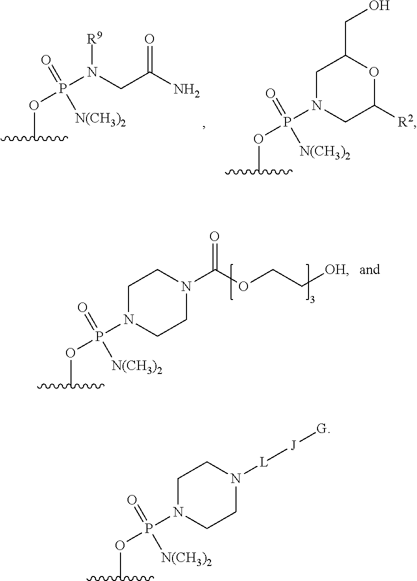

In some embodiments of the conjugate of formula (I), A′ is selected from

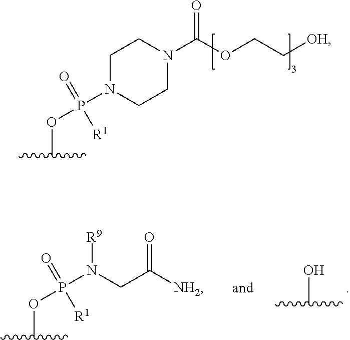

In some embodiments of the conjugate of formula (I), A′ is selected from

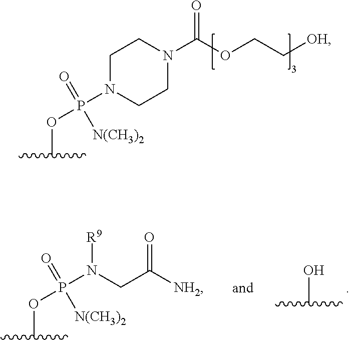

In some embodiments of the conjugate of formula (I), A′ is —OH. In some embodiments, A′ is

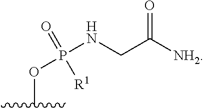

In some embodiments, A′ is

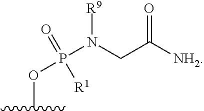

In some embodiments, A′ is

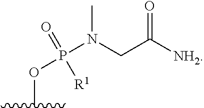

In some embodiments, A′ is

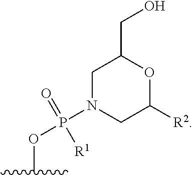

In some embodiments, A′ is

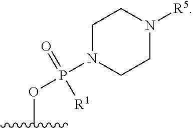

In some embodiments, A′ is

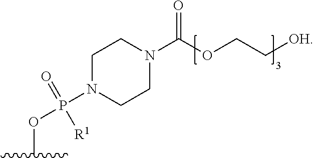

In some embodiments, A′ is

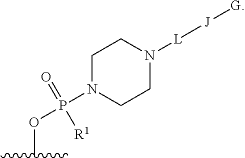

In some embodiments of the conjugate of formula (I), A′ is

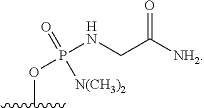 [SMILES: *OP(=O)(NCC(N)=O)N(C)C]

In some embodiments, A′ is

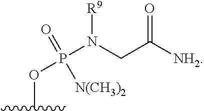

In some embodiments, A′ is

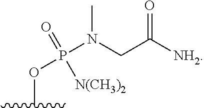 [SMILES: *OP(=O)(N(C)C)N(C)CC(N)=O]

In some embodiments, A′ is

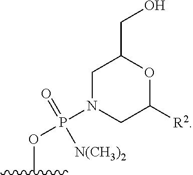

In some embodiments, A′ is

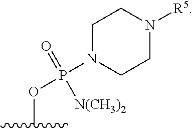

In some embodiments, A′ is

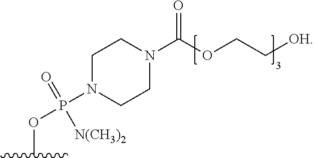

In some embodiments, A′ is

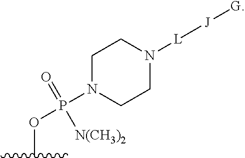

In some embodiments of the conjugate of formula (I), R⁵ is —C(O)(OCH₂CH₂)ₓ—OH, wherein x is 3-10. In some embodiments, R⁵ is

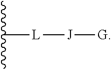

In some embodiments, R⁵ is —C(O)(OCH₂CH₂)ₓ—OH, wherein x is 3-10, or R⁵ is

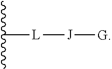

In some embodiments of the conjugate of formula (I), R⁹ is methyl. In some embodiments, R⁹ is ethyl. In some embodiments, R⁹ is n-propyl. In some embodiments, R⁹ is isopropyl.

In some embodiments of the conjugate of formula (I), R¹ is —OH. In some embodiments, each R¹ is —OH. In some embodiments, R¹ is —N(R³)(R⁴). In some embodiments, each R¹ is —N(R³)(R⁴). In some embodiments, each R³ is —H. In some embodiments, each R³ is —C₁₋₆-alkyl. In some embodiments, each R⁴ is —H. In some embodiments, each R⁴ is —C₁₋₆-alkyl. In some embodiments, R¹ is —N(CH₃)₂. In some embodiments, each R¹ is —N(CH₃)₂.

In some embodiments of the conjugate of formula (I), R² is —H. In some embodiments, R² is a nucleobase comprising a ring selected from pyridine, pyrimidine, purine, and deaza-purine. In some embodiments, each R² is a nucleobase comprising a ring selected from pyridine, pyrimidine, purine, and deaza-purine. In some embodiments, R² is a nucleobase functionalized with a chemical protecting group, wherein the nucleobase comprises a ring selected from pyridine, pyrimidine, purine, and deaza-purine. In some embodiments, each R² is a nucleobase functionalized with a chemical protecting group, wherein the nucleobase comprises a ring selected from pyridine, pyrimidine, purine, and deaza-purine. In some embodiments, each R² is selected from the group consisting of adenine, guanine, cytosine, 5-methyl-cytosine, thymine, uracil, and hypoxanthine. In some embodiments, each R² is selected from the group consisting of adenine, guanine, cytosine, thymine, and uracil.

In some embodiments of the conjugate of formula (I), t is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In some embodiments, t is an integer between 10 and 30. In some embodiments, t is an integer between 18 and 30. In some embodiments, t is an integer between 13 and 33. In some embodiments, t is an integer between 15 and 35. In some embodiments, t is 18. In some embodiments, t is 19. In some embodiments, t is 20. In some embodiments, t is 21. In some embodiments, t is 22. In some embodiments, t is 23. In some embodiments, t is 24. In some embodiments, t is 25. In some embodiments, t is 26. In some embodiments, t is 27. In some embodiments, t is 28. In some embodiments, t is 29. In some embodiments, t is 30.

In some embodiments of the conjugate of formula (I), each R² is a nucleobase, and all R² groups taken together form a targeting sequence. In some embodiments, the targeting sequence is complementary to one or more bases of exon 11 in the human LMNA gene including the wild-type sequence (SEQ ID NO:1) and/or the sequence found in HGPS patients, as shown in SEQ ID NO: 2. These target sequences are shown in Table 1 below:

TABLE 1
Exemplary LMNA target sequences

| | | SEQ ID |
|---|---|---|
| NAME | SEQUENCE | NO: |
| LMNA | GGCTCCCACTGCAGCAGCTCGGGGGACC | 1 |
| exon 11 | CCGCTGAGTACAACCTGCGCTCGCGCAC | |
| | CGTGCTGTGCGGGACCTGCGGGCAGCCT | |
| | GCCGACAAGGCATCTGCCAGCGGCTCAG | |
| | GAGCCCAGGTGGGCGGACCCATCTCCTC | |
| | TGGCTCTTCTGCCTCCAGTGTCACGGTCA | |
| | CTCGCAGCTACCGCAGTGTGGGGGGCAG | |
| | TGGGGGTGGCAGCTTCGGGGACAATCTG | |
| | GTCACCCGCTCCTACCTCCTGGGCAACTC | |
| | CAGCCCCCGAACCCAG | |
| HGPS | GGCTCCCACTGCAGCAGCTCGGGGGACC | 2 |
| exon 11 | CCGCTGAGTACAACCTGCGCTCGCGCAC | |
| | CGTGCTGTGCGGGACCTGCGGGCAGCCT | |
| | GCCGACAAGGCATCTGCCAGCGGCTCAG | |
| | GAGCCCAGGTGGGTGGACCCATCTCCTC | |
| | TGGCTCTTCTGCCTCCAGTGTCACGGTCA | |
| | CTCGCAGCTACCGCAGTGTGGGGGGCAG | |
| | TGGGGGTGGCAGCTTCGGGGACAATCTG | |
| | GTCACCCGCTCCTACCTCCTGGGCAACTC | |
| | CAGCCCCCGAACCCAG | |

Examples include targeting sequences that are fully complementary to LMNA exon 11 (SEQ ID NO:1 or 2) including those that are also complementary to the cryptic splice site of LMNA exon 11 underlined in SEQ ID NO:1 and 2 in Table 1 (e.g., CAGGTGGGC/T).

In certain embodiments, the degree of complementarity between the target and antisense targeting sequence is sufficient to form a stable duplex. The region of complementarity of the antisense oligomers with the target RNA sequence may be as short as 8-11 bases, but is preferably 12-15 bases or more, e.g., 12-20 bases, 12-25, or 15-25 bases, including all integers and ranges in between these ranges. An antisense oligomer of about 14-15 bases is generally long enough to have a unique complementary sequence in the target mRNA. In certain embodiments, a minimum length of complementary bases may be required to achieve the requisite binding T_{M}.

The stability of the duplex formed between an oligomer and a target sequence is a function of the binding T_{M} and the susceptibility of the duplex to cellular enzymatic cleavage. The T_{M} of an oligomer with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers may have a binding T_{M}, with respect to a complementary-sequence RNA, of greater than body temperature and, in some embodiments greater than about 45° C. or 50° ° C. Ty's in the range 60-80° C. or greater are also included. According to well-known principles, the T_{M} of an oligomer, with respect to a complementary-based RNA hybrid, can be increased by increasing the ratio of C:G paired bases in the duplex, or by increasing the length (in base pairs) of the heteroduplex, or both. At the same time, for purposes of optimizing cellular uptake, it may be advantageous to limit the size of the oligomer. For this reason, compounds of the disclosure include compounds that show a high T_{M} (45-50° C. or greater) at a length of 25 bases or less.

In some embodiments, the antisense oligonucleotides contain base modifications or substitutions. For example, certain nucleobases may be selected to increase the binding affinity of the antisense oligonucleotides described herein. These include 5-substituted pyrimidines, 6-azapyrimidines and N-2, N-6 and 0-6 substituted purines, including 2-aminopropyladenine, 5-propynyluracil, 5-propynylcytosine and 2,6-diaminopurine. 5-methylcytosine substitutions have been shown to increase nucleic acid duplex stability by 0.6-1.2° ° C., and may be incorporated into the antisense oligonucleotides described herein. In one embodiment, at least one pyrimidine base of the oligonucleotide comprises a 5-substituted pyrimidine base, wherein the pyrimidine base is selected from the group consisting of cytosine, thymine and uracil. In one embodiment, the 5-substituted pyrimidine base is 5-methylcytosine. In another embodiment, at least one purine base of the oligonucleotide comprises an N-2, N-6 substituted purine base. In one embodiment, the N-2, N-6 substituted purine base is 2, 6-diaminopurine.

Provided in Table 2 are various embodiments of nucleotide moieties as described herein.

TABLE 2
Various embodiments of nucleotide moieties

| | |
|---|---|
| 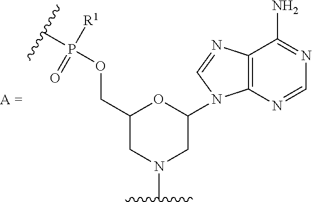 | 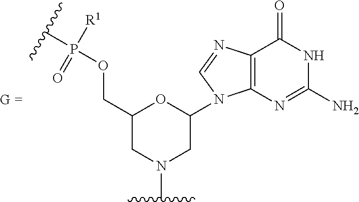 |
| 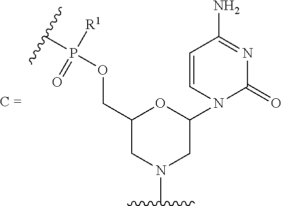 | 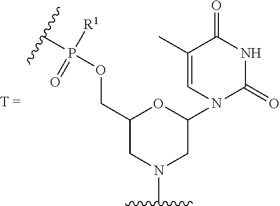 |
| 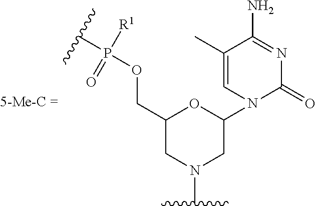 | 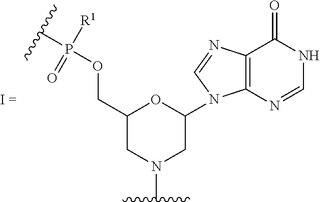 |
| 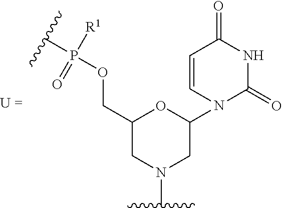 | |

In certain embodiments, oligomers as long as 40 bases may be suitable, where at least a minimum number of bases, e.g., 10-12 bases, are complementary to the target sequence. In general, however, facilitated or active uptake in cells is optimized at oligomer lengths less than about 30. Contemplated herein are antisense oligomers that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous and/or non-contiguous bases are complementary to a target sequence described herein, including the target sequences of SEQ ID NOs: 1 and/or 2, or variants thereof.

In certain embodiments, antisense oligomers may be 100% complementary to the LMNA pre-mRNA nucleic acid target sequence, or they may include mismatches, e.g., to accommodate variants, as long as a heteroduplex formed between the oligomer and the target sequence is sufficiently stable to withstand the action of cellular nucleases and other modes of degradation or displacement which may occur in vivo. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, such that a biological activity of the nucleic acid target, e.g., expression of the progerin protein(s), is modulated.

In certain embodiments, the antisense activity of an oligomer may be enhanced by using a mixture of uncharged and cationic phosphorodiamidate linkages. The total number of cationic linkages in the oligomer can vary from 1 to 10 (including all integers in between), and be interspersed throughout the oligomer. In some embodiments, the number of charged linkages is at least 2 and no more than half the total backbone linkages, e.g., between 2, 3, 4, 5, 6, 7, or 8 positively charged linkages, and preferably each charged linkage is separated along the backbone by at least 1, 2, 3, 4, or 5 uncharged linkages.

Exemplary antisense sequences for targeting the human LMNA pre-mRNA are shown in Table 3 below. Antisense oligonucleotides can comprise all or a portion of these targeting sequences.

TABLE 3
Exemplary HGPS targeting sequences

| Sequence | | SEQ ID |
|---|---|---|
| name | Targeting Sequence 5′-3′ | NO: |
| Ex11-1 | CTGAGCCGCTGGCAGATGCCTTGTC | 3 |
| Ex11-2 | GAGGAGATGGGTCCACCCACCTGGG | 4 |

Accordingly, in some embodiments of the conjugate of formula (I), each R² is a nucleobase, and all R² groups taken together form a targeting sequence, wherein the targeting sequence is selected from: (a) SEQ ID NO: 3 (CTGAGCCGCTGGCAGATGCCTTGTC) wherein t is 23; and (b) SEQ ID NO: 4 (GAGGAGATGGGTCCACCCACCTGGG) wherein t is 23.

In some embodiments of the conjugate of formula (I), each R² is a nucleobase, and all R² groups taken together form a targeting sequence, wherein the targeting sequence is SEQ ID NO: 3 (CTGAGCCGCTGGCAGATGCCTTGTC) wherein t is 23. In some embodiments, each R² is a nucleobase, and all R² groups taken together form a targeting sequence, wherein the targeting sequence has at least 80%, at least 85%, at least 90%, at least 95%, at least 97%, at least 98%, or at least 99% sequence identity or sequence homology to SEQ ID NO: 3 (CTGAGCCGCTGGCAGATGCCTTGTC).

In some embodiments of the conjugate of formula (I), each R² is a nucleobase, and all R² groups taken together form a targeting sequence, wherein the targeting sequence is SEQ ID NO: 4 (GAGGAGATGGGTCCACCCACCTGGG) wherein t is 23. In some embodiments, each R² is a nucleobase, and all R² groups taken together form a targeting sequence, wherein the targeting sequence has at least 80%, at least 85%, at least 90%, at least 95%, at least 97%, at least 98%, or at least 99% sequence identity or sequence homology to

SEQ ID NO: 4
(GAGGAGATGGGTCCACCCACCTGGG).

In some embodiments of the conjugate of formula (I), E′ is selected from —H, —C₁₋₆-alkyl, —C(O)C₁₋₆-alkyl, benzoyl, stearoyl, trityl, monomethoxytrityl, dimethoxytrityl, and trimethoxytrityl. In some embodiments, E′ is selected from —H, —C₁₋₆-alkyl, —C(O)C₁₋₆-alkyl, benzoyl, stearoyl, trityl, monomethoxytrityl, dimethoxytrityl, trimethoxytrityl, and

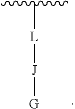

In some embodiments of the conjugate of formula (I), E′ is selected from —H, —C(O)CH₃, benzoyl, stearoyl, trityl, and 4-methoxytrityl. In some embodiments, E′ is selected from —H, —C(O)CH₃, benzoyl, stearoyl, trityl, 4-methoxytrityl, and

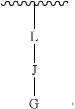

In some embodiments of the conjugate of formula (I), E′ is —H. In some embodiments, E′ is —C₁₋₆-alkyl. In some embodiments, E′ is —C(O)C₁₋₆-alkyl. In some embodiments, E′ is —C(O)CH₃. In some embodiments, E′ is benzoyl. In some embodiments, E′ is stearoyl. In some embodiments, E′ is trityl. In some embodiments, E′ is monomethoxytrityl. In some embodiments, E′ is dimethoxytrityl. In some embodiments, E′ is trimethoxytrityl. In some embodiments, E′ is 4-methoxytrityl. In some embodiments, E′ is

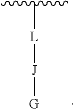

In some embodiments of the conjugate of formula (I), A′ is selected from

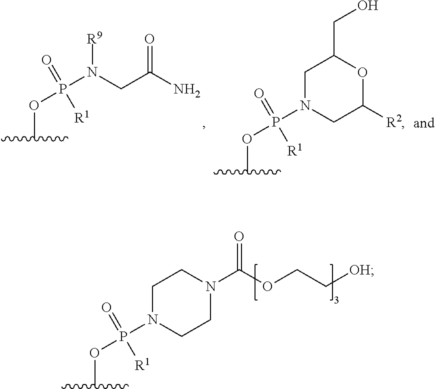

### and E′ is

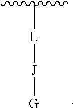

In some embodiments of the conjugate of formula (I), A′ is selected from

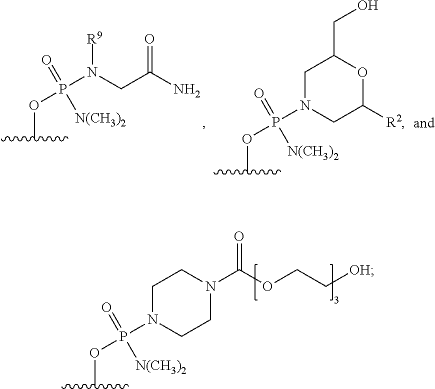

### and E′ is

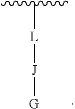

In some embodiments of the conjugate of formula (I), A′ is

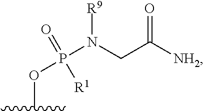

### and E′ is

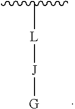

In some embodiments of the conjugate of formula (I), A′ is

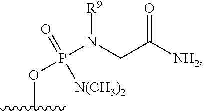

### and E′ is

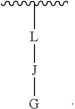

In some embodiments of the conjugate of formula (I), A′ is

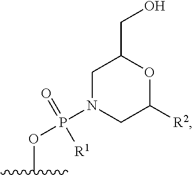

### and E′ is

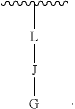

In some embodiments of the conjugate of formula (I), A′ is

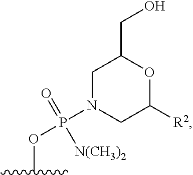

### and E′ is

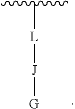

In some embodiments of the conjugate of formula (I), A′ is

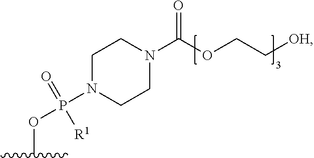

### and E′ is

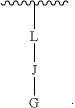

In some embodiments of the conjugate of formula (I), A′ is

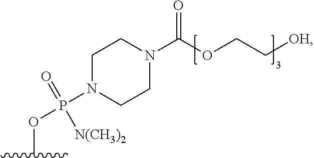

### and E′ is

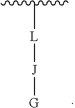

In some embodiments of the conjugate of formula (I), A′ is

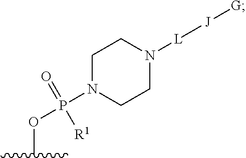

and E′ is selected from H, —C(O)CH₃, trityl, 4-methoxytrityl, benzoyl, and stearoyl.

In some embodiments of the conjugate of formula (I), A′ is

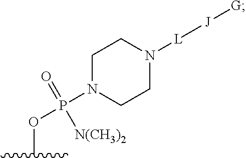

and E′ is selected from H, —C(O)CH₃, trityl, 4-methoxytrityl, benzoyl, and stearoyl.

Variable L of the conjugate of formula (I) may be any naturally occurring amino acid or non-natural amino acid (e.g., a β- or γ-amino acid). Accordingly, in some embodiments, L is alanine, arginine, asparagine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In other embodiments, L is isoglutamine, γ-aminobutyric acid, beta-alanine, 6-aminohexanoic acid, 6-aminopentanoic acid, norleucine, norvaline, p-fluorophenylalanine, or ethionine. In some embodiments of the conjugate of formula (I), L is glutamic acid, isoglutamine, glycine, proline, γ-aminobutyric acid, or β-alanine. In some embodiments, L is glycine, proline, or β-alanine. In some embodiments, L is glutamic acid. In some embodiments, L is glutamic acid, provided that —COOH, if present in the glutamic acid residue, is replaced with —CONH₂. In some embodiments, L is isoglutamine. In some embodiments, L is glycine. In some embodiments, L is proline. In some embodiments, L is γ-aminobutyric acid. In some embodiments, L is β-alanine.

Variable J of the conjugate of formula (I) may be any cell-penetrating peptide (referred to herein as “CPP”) known in the art.

The CPP has the capability of inducing cell penetration within 30%, 40%, 50%, 60%, 70%, 80%, 90% or 100% of cells of a given cell culture population, including all integers in between, and allow macromolecular translocation within multiple tissues in vivo upon systemic administration. In some embodiments, the CPP is an arginine-rich peptide. The term “arginine-rich” refers to a CPP having at least 2, and preferably 2, 3, 4, 5, 6, 7, or 8 arginine residues, each optionally separated by one or more uncharged, hydrophobic residues, and optionally containing about 6-14 amino acid residues. In some embodiments, the CPP is linked at its carboxy terminus to the 3′ and/or 5′ end of the antisense oligonucleotide via the linking amino acid, L, and the CPP is capped at its amino terminus by substituent G.

TABLE 4
Exemplary CPPs (SEQ ID NOS: 5 - 21) and CPP and
linker moiety combinations (SEQ ID NOS: 22-25)

| NAME | | SEQ |
|---|---|---|
| (DESIGNATION) | SEQUENCE | ID NO. |
| rTAT | RRRQRRKKR | 5 |
| Tat | RKKRRQRRR | 6 |
| R₉F₂ | RRRRRRRRRFF | 7 |
| R₅F₂R₄ | RRRRRFFRRRR | 8 |
| R₄ | RRRR | 9 |
| R₅ | RRRRR | 10 |
| R₆ | RRRRRR | 11 |
| R₇ | RRRRRRR | 12 |
| R₈ | RRRRRRRR | 13 |
| R₉ | RRRRRRRRR | 14 |
| (RX)₈ | RXRXRXRXRXRXRXRX | 15 |
| (RXR)₄ | RXRRXRRXRRXR | 16 |
| (RXR)₅ | RXRRXRRXRRXRRXR | 17 |
| (RXRRBR)₂ | RXRRBRRXRRBR | 18 |
| (RAR)₄F₂ | RARRARRARRARFF | 19 |
| (RGR)₄F₂ | RGRRGRRGRRGRFF | 20 |
| (RFF)₃R | RFFRFFRFFR | 21 |
| (RXR)₄XB | RXRRXRRXRRXRXB | 22 |
| (RFF)₃RXB | RFFRFFRFFRXB | 23 |
| (RFF)₃RG | RFFRFFRFFRG | 24 |
| R₆G | RRRRRRG | 25 |
| (RBR)₂FQILYBRBR | RBRRBRFQILYBRBR | 26 |
| (RBR)₂FQILYRBHBH | RBRRBRFQILYRBHBH | 27 |

X is 6-aminohexanoic acid; B is ß-alanine; A is alanine; F is phenylalanine; G is glycine; R is arginine; Q is glutamine; K is lysine; I is isoleucine; L is leucine; Y is tyrosine; H is histidine.

The transport moieties as described above have been shown to greatly enhance cell entry of attached oligomers, relative to uptake of the oligomer in the absence of the attached transport moiety. Uptake may be enhanced at least ten fold, and, in some embodiments, twenty fold, relative to the unconjugated compound.

The use of arginine-rich peptide transporters (i.e., cell-penetrating peptides) are particularly useful in practicing the present disclosure. Certain peptide transporters have been shown to be highly effective at delivery of antisense compounds into primary cells including muscle cells. Furthermore, compared to other known peptide transporters such as Penetratin and the Tat peptide, the peptide transporters described herein, when conjugated to an antisense PMO, demonstrate an enhanced ability to alter splicing of several gene transcripts.

CPPs, their synthesis, and methods of conjugating to an oligomer are further described in U.S. Application Publication No. 2012/0289457 and PCT Patent Application Publication Nos. WO 2004/097017, WO 2009/005793, and WO 2012/150960, the disclosures of which are incorporated herein by reference in their entirety.

In some embodiments, J comprises or is selected from SEQ ID NOS: 5-21. In some embodiments, J comprises or is selected from SEQ ID NOS: 22-25. In some embodiments, J comprises or is selected from SEQ ID NOS: 26-27. In certain embodiments, J is SEQ ID NO: 11. In some embodiments, J is SEQ ID NO: 25. In some embodiments, J is SEQ ID NO: 26. In some embodiments, J is SEQ ID NO: 27.

In some embodiments of the conjugate of formula (I), G is selected from —H, —C(O)CH₃, benzoyl, and stearoyl. In some embodiments, G is —H or —C(O)C₁₋₆-alkyl. In some embodiments, G is —H or —C(O)CH₃. In some embodiments, G is H. In some embodiments, G is —C(O)C₁₋₆-alkyl. In some embodiments, G is-C(O)CH₃. In some embodiments, G is benzoyl. In some embodiments, G is stearoyl.

In some embodiments,

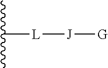

has the following structure:

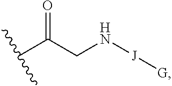

wherein J is selected from SEQ ID NOS: 1-27, and wherein G is selected from —H, —C(O)CH₃, benzoyl, and stearoyl. In some embodiments, J is SEQ ID NO: 11. In some embodiments, J is SEQ ID NO: 25. In some embodiments, J is SEQ ID NO: 26. In some embodiments, J is SEQ ID NO: 27. In some embodiments, G is —H. In some embodiments, G is —C(O)CH₃.

In some embodiments,

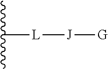

has the following structure:

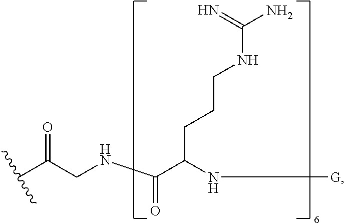

wherein G is selected from —H, —C(O)CH₃, benzoyl, and stearoyl. In some embodiments, G is —H. In some embodiments, G is —C(O)CH₃.

In some embodiments,

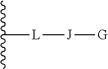

has the following structure:

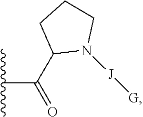

wherein J is selected from SEQ ID NOS: 1-27, and wherein G is selected from —H, —C(O)CH₃, benzoyl, and stearoyl. In some embodiments, J is SEQ ID NO: 11. In some embodiments, J is SEQ ID NO: 25. In some embodiments, J is SEQ ID NO: 26. In some embodiments, J is SEQ ID NO: 27. In some embodiments, G is —H. In some embodiments, G is —C(O)CH₃.

In some embodiments,

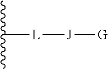

has the following structure:

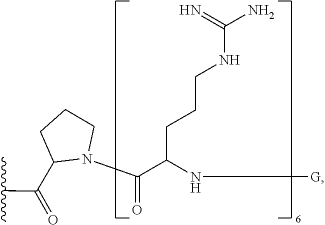

wherein G is selected from —H, —C(O)CH₃, benzoyl, and stearoyl. In some embodiments, G is —H. In some embodiments, G is —C(O)CH₃.

In some embodiments,

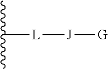

has the following structure:

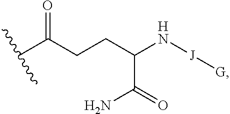

wherein J is selected from SEQ ID NOS: 1-27, and wherein G is selected from —H, —C(O)CH₃, benzoyl, and stearoyl. In some embodiments, J is SEQ ID NO: 11. In some embodiments, J is SEQ ID NO: 25. In some embodiments, J is SEQ ID NO: 26. In some embodiments, J is SEQ ID NO: 27. In some embodiments, G is —H. In some embodiments, G is —C(O)CH₃.

In some embodiments,

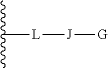

has the following structure:

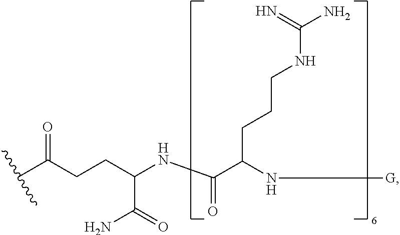

wherein G is selected from —H, —C(O)CH₃, benzoyl, and stearoyl. In some embodiments, G is —H. In some embodiments, G is —C(O)CH₃.

In some embodiments,

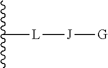

has the following structure:

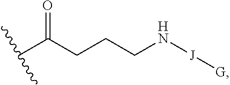

wherein J is selected from SEQ ID NOS: 1-27, and wherein G is selected from —H, —C(O)CH₃, benzoyl, and stearoyl. In some embodiments, J is SEQ ID NO: 11. In some embodiments, J is SEQ ID NO: 25. In some embodiments, J is SEQ ID NO: 26. In some embodiments, J is SEQ ID NO: 27. In some embodiments, G is —H. In some embodiments, G is —C(O)CH₃.

In some embodiments,

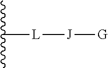

has the following structure:

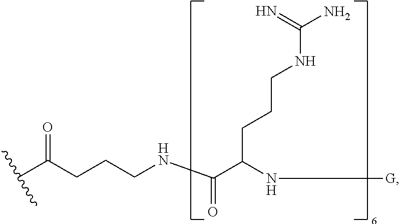

wherein G is selected from —H, —C(O)CH₃, benzoyl, and stearoyl. In some embodiments, G is —H. In some embodiments, G is —C(O)CH₃.

In some embodiments,

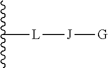

has the following structure:

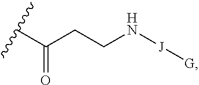

wherein J is selected from SEQ ID NOS: 1-27, and wherein G is selected from —H, —C(O)CH₃, benzoyl, and stearoyl. In some embodiments, J is SEQ ID NO: 11. In some embodiments, J is SEQ ID NO: 25. In some embodiments, J is SEQ ID NO: 26. In some embodiments, J is SEQ ID NO: 27. In some embodiments, G is —H. In some embodiments, G is —C(O)CH₃.

In some embodiments,

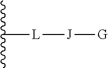

has the following structure:

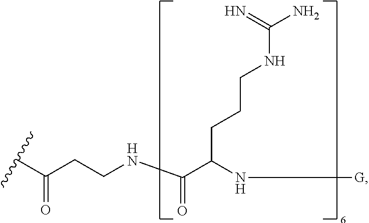

wherein G is selected from —H, —C(O)CH₃, benzoyl, and stearoyl. In some embodiments, G is —H. In some embodiments, G is —C(O)CH₃.

In some embodiments, the antisense oligomer conjugate of formula (I) has a structure according to formula (IA):

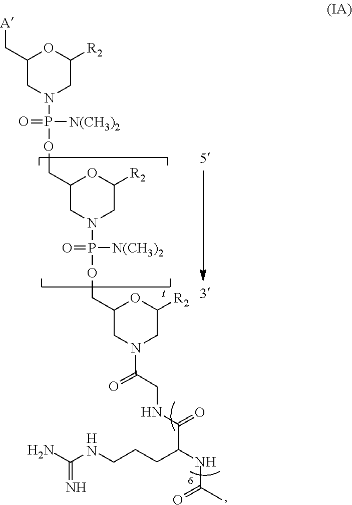

or a pharmaceutically acceptable salt thereof.

In some embodiments of the antisense oligomer conjugate of formula (IA), A′ is a moiety selected from

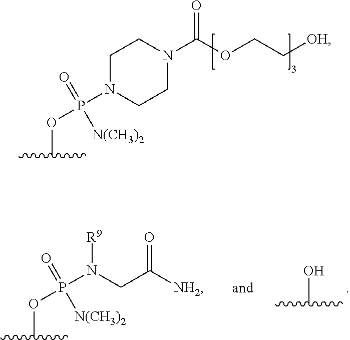

some embodiments, A′ is

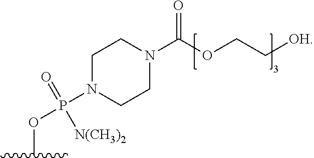

In some embodiments, A′ is

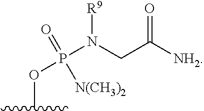

In some embodiments, A′ is

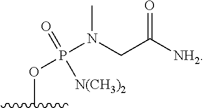 [SMILES: *OP(=O)(N(C)C)N(C)CC(N)=O]

In some embodiments, A′ is

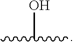 [SMILES: *O]

In some embodiments of the antisense oligomer conjugate of formula (IA), each R² is a nucleobase, and all R² groups taken together form a targeting sequence, wherein the targeting sequence is SEQ ID NO: 3 (CTGAGCCGCTGGCAGATGCCTTGTC) wherein t is 23. In some embodiments of the conjugate of formula (LA), each R² is a nucleobase, and all R² groups taken together form a targeting sequence, wherein the targeting sequence is SEQ ID NO: 4 (GAGGAGATGGGTCCACCCACCTGGG) wherein t is 23.

In some embodiments, the antisense oligomer conjugate of formula (I) has a structure according to formula (II):

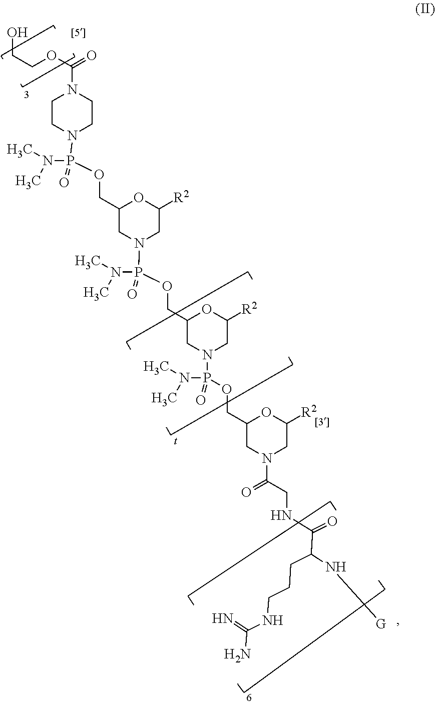

or a pharmaceutically acceptable salt thereof.

In some embodiments of the antisense oligomer conjugate of formula (II), each R² is a nucleobase, and all R² groups taken together form a targeting sequence, wherein the targeting sequence is SEQ ID NO: 3 (CTGAGCCGCTGGCAGATGCCTTGTC) wherein t is 23. In some embodiments of the conjugate of formula (II), each R² is a nucleobase, and all R² groups taken together form a targeting sequence, wherein the targeting sequence is SEQ ID NO: 4 (GAGGAGATGGGTCCACCCACCTGGG) wherein t is 23.

In some embodiments, the antisense oligomer conjugate of formula (I) has a structure according to formula (IIA):

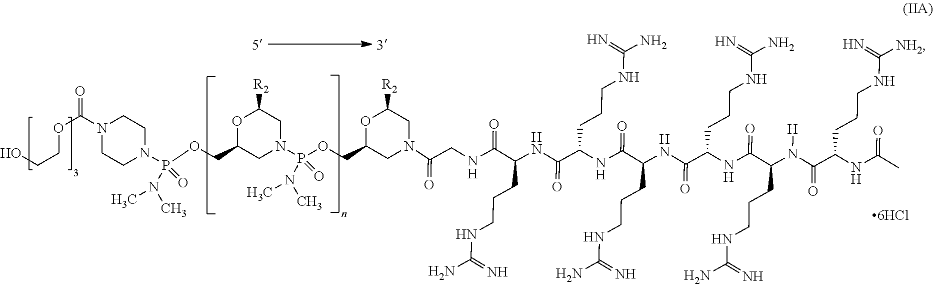

wherein n is 9-39.

In some embodiments of the antisense oligomer conjugate of formula (IIA), each R² is a nucleobase, and all R² groups taken together form a targeting sequence, wherein the targeting sequence is SEQ ID NO: 3 (CTGAGCCGCTGGCAGATGCCTTGTC) wherein n is 24. In some embodiments of the conjugate of formula (IIA), each R² is a nucleobase, and all R² groups taken together form a targeting sequence, wherein the targeting sequence is SEQ ID NO: 4 (GAGGAGATGGGTCCACCCACCTGGG) wherein n is 24.

In some embodiments, the antisense oligomer conjugate of formula (I) has a structure according to formula (IIIa):

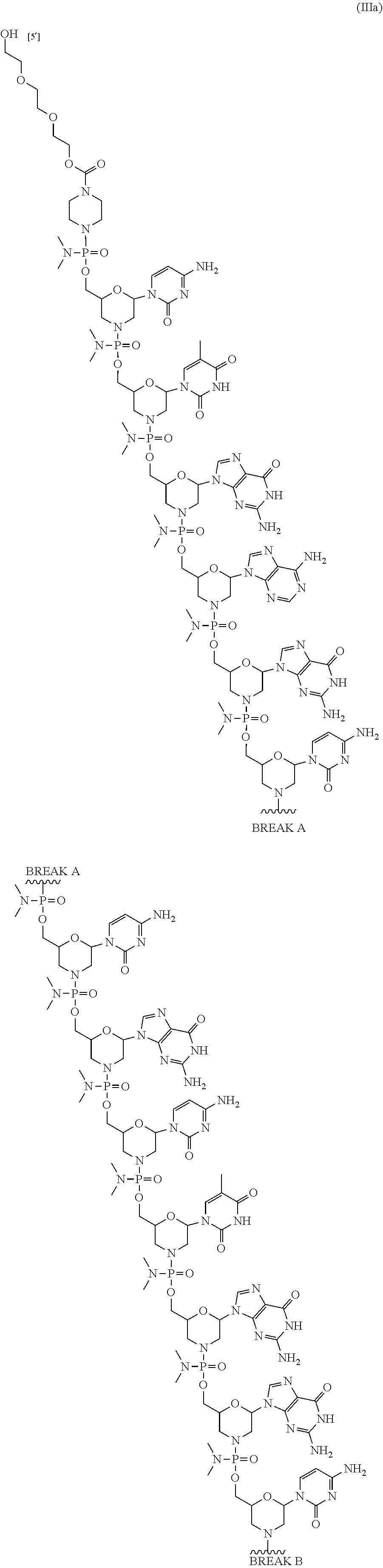

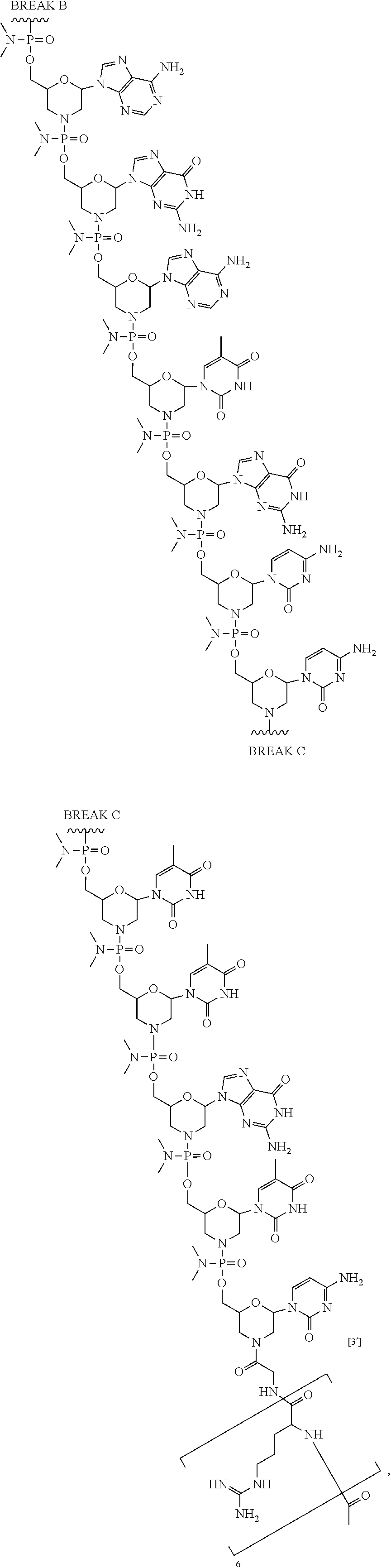

or a pharmaceutically acceptable salt thereof.

In some embodiments, the antisense oligomer conjugate of formula (I) has a structure according to formula (IIIb):

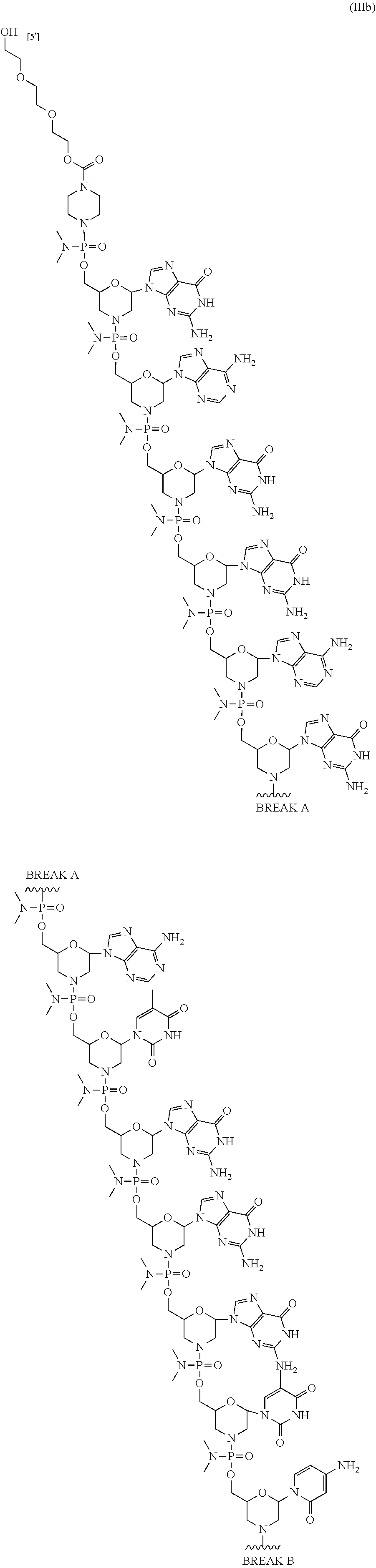

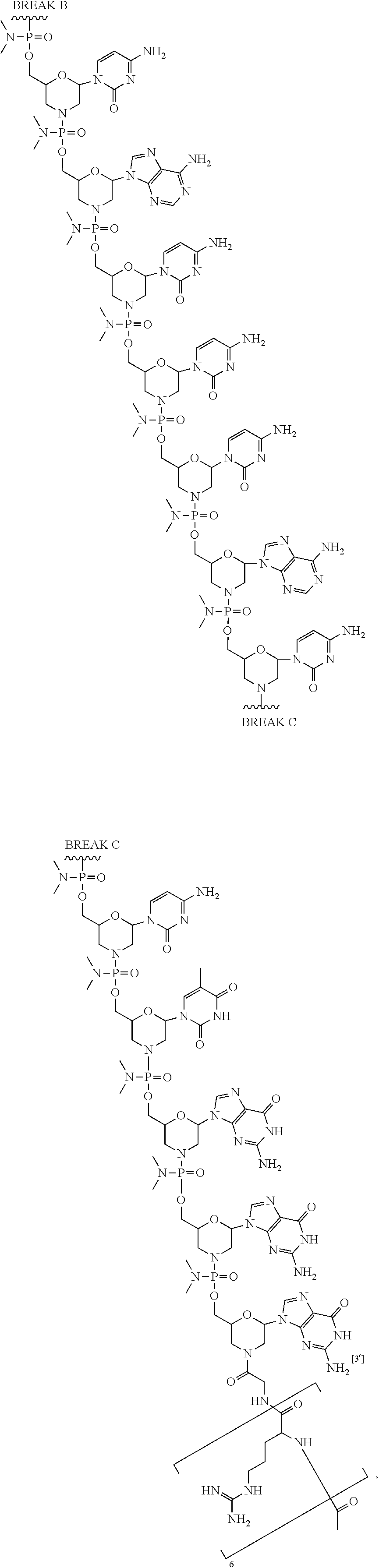

or a pharmaceutically acceptable salt thereof.

In some embodiments, the antisense oligomer conjugate of formula (I) has a structure according to formula (IVa):

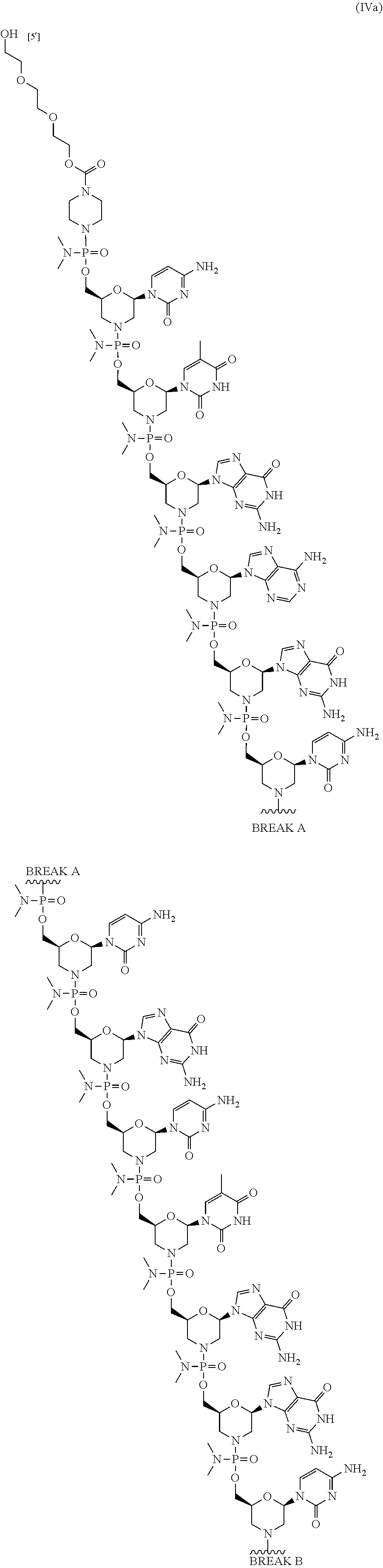

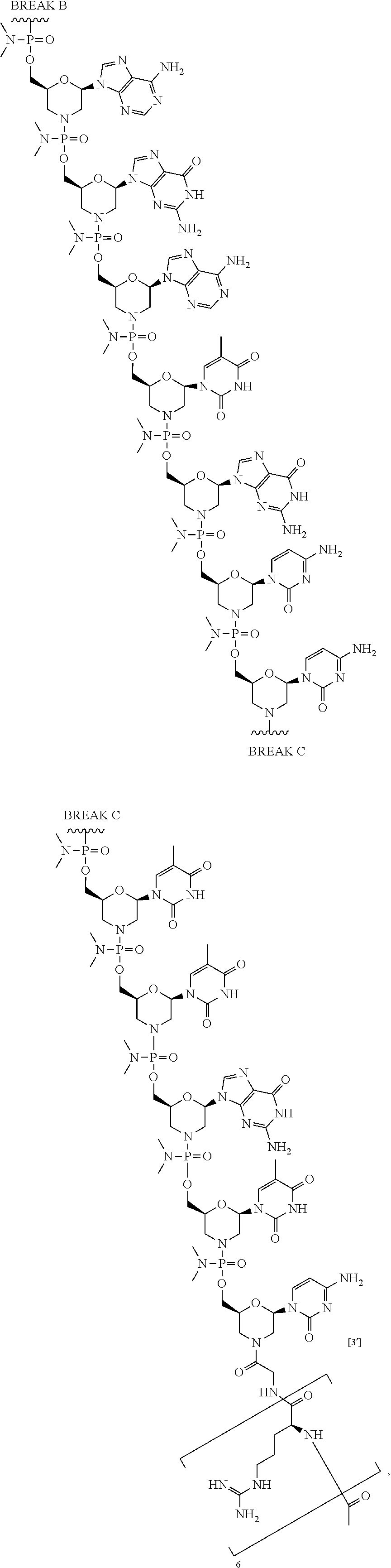

or a pharmaceutically acceptable salt thereof.

In some embodiments, the antisense oligomer conjugate of formula (I) has a structure according to formula (IVb):

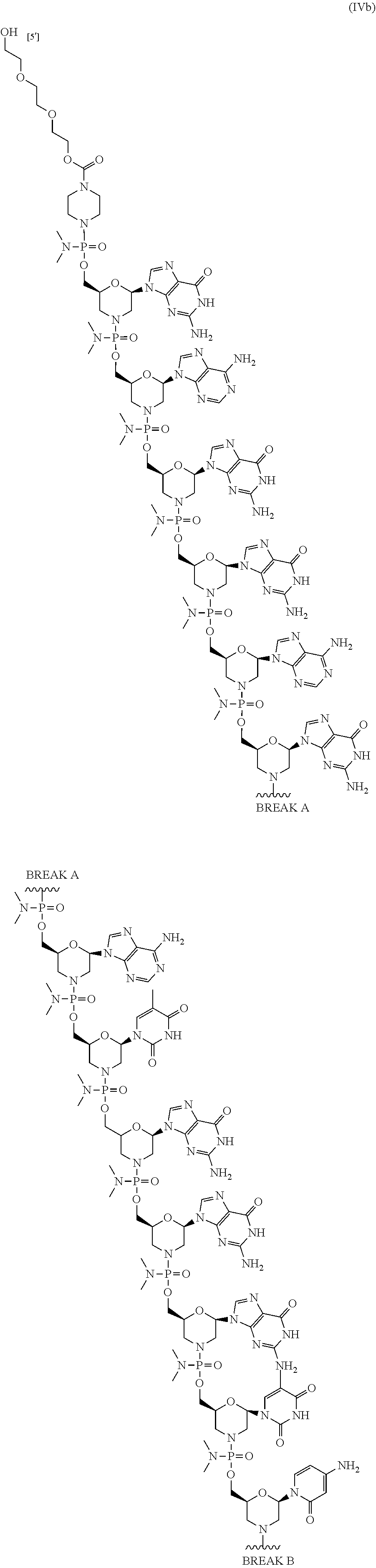

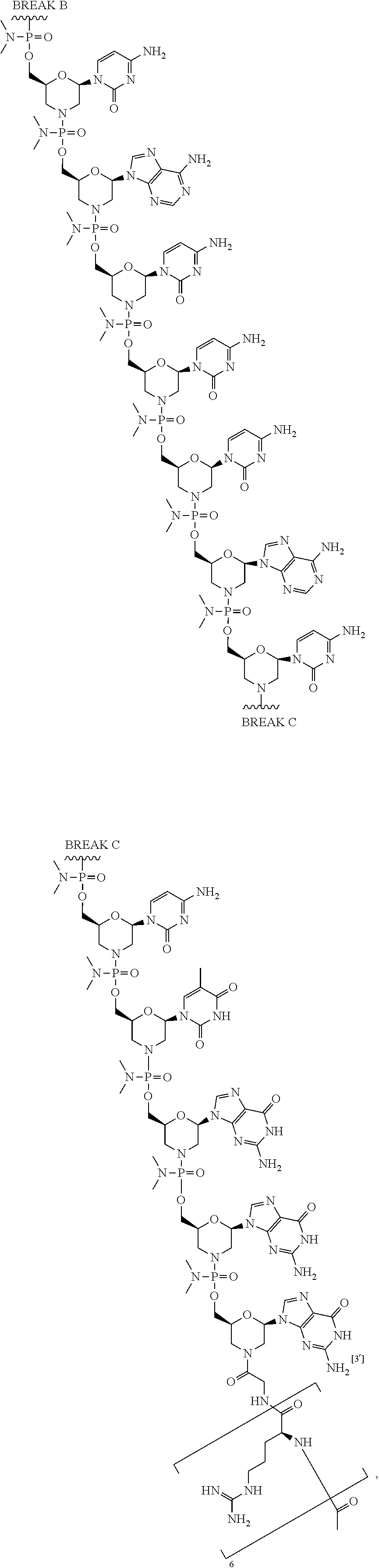

or a pharmaceutically acceptable salt thereof.

For clarity, the structural formulas of the antisense oligomer conjugates of formulas (IIIa), (IIIb), (IVa), and (IVb) are continuous structural formulas from 5′ to 3′, and, for ease of illustrating the entire structural formula in a compact form, are presented herein with various illustration breaks labeled “BREAK A,” “BREAK B,” and “BREAK C.” The skilled artisan would understand that, for example, each indication of “BREAK A” shows a continuation of the illustration of the structural formula at these points. The same is true for each instance of “BREAK B” and “BREAK C” in the structural formula of the antisense oligomers of formulas (IIIa), (IIIb), (IVa), and (IVb). None of the illustration breaks described above or used herein are intended to indicate, nor would the skilled artisan understand them to mean, an actual discontinuation of the structural formula of the antisense oligomer conjugates of formulas (IIIa), (IIIb), (IVa), and (IVb).

In some embodiments, the antisense oligomer conjugate of the disclosure including, for example, a conjugate of formula (I), (IA), (II), (IIIa), (IIIb), (IVa), and (IVb), is a pharmaceutically acceptable salt. In certain embodiments, the pharmaceutically acceptable salt is an HCl (hydrochloric acid) salt. For example, in some embodiments, the conjugate of formula (I) is an HCl salt. In certain embodiments, the conjugate of formula (I) is a 6HCl salt. In some embodiments, the conjugate of formula (IA) is an HCl salt. In certain embodiments, the conjugate of formula (IA) is a 6HCl salt. In some embodiments, the conjugate of formula (II) is an HCl salt. In certain embodiments, the conjugate of formula (II) is a 6HCl salt. In some embodiments, the conjugate of formula (IIIa) is an HCl salt. In certain embodiments, the conjugate of formula (IIIa) is a 6HCl salt. In some embodiments, the conjugate of formula (IIIb) is an HCl salt. In certain embodiments, the conjugate of formula (IIIb) is a 6HCl salt. In some embodiments, the conjugate of formula (IVa) is an HCl salt. In certain embodiments, the conjugate of formula (IVa) is a 6HCl salt. In some embodiments, the conjugate of formula (IVb) is an HCl salt. In certain embodiments, the conjugate of formula (IVb) is a 6HCl salt.

In some embodiments, the antisense oligomer conjugate is present in the pharmaceutical composition at a concentration between about 10 mg/mL and about 125 mg/mL. In some embodiments, the antisense oligomer conjugate is present in the pharmaceutical composition at a concentration between about 50 mg/mL and about 125 mg/mL. In some embodiments, the antisense oligomer conjugate is present in the pharmaceutical composition at a concentration between about 75 mg/mL and about 125 mg/mL. In some embodiments, the antisense oligomer conjugate is present in the pharmaceutical composition at a concentration between about 90 mg/mL and about 110 mg/mL. In some embodiments, the antisense oligomer conjugate is present in the pharmaceutical composition at a concentration of about 100 mg/mL.

### Excipients, Diluents, and Carriers

In addition to an antisense oligomer conjugate of formula (I), as described herein, the pharmaceutical compositions of the present disclosure comprise benzyl alcohol. The pharmaceutical compositions may further comprise additional excipients, diluents, or carriers.

In some embodiments, the benzyl alcohol is present in a range from about 0.1% weight by volume to about 10% weight by volume, more preferably from about 0.5% weight by volume to about 7% weight by volume, more preferable from about 0.5% weight by volume to about 5% weight by volume. In some embodiments, pharmaceutical composition comprises about 0.5% weight by volume benzyl alcohol. In some embodiments, pharmaceutical composition comprises about 1% weight by volume benzyl alcohol. In some embodiments, pharmaceutical composition comprises about 1.5% weight by volume benzyl alcohol. In some embodiments, pharmaceutical composition comprises about 2.0% weight by volume benzyl alcohol. In some embodiments, pharmaceutical composition comprises about 2.5% weight by volume benzyl alcohol. In some embodiments, pharmaceutical composition comprises about 3.0% weight by volume benzyl alcohol. In some embodiments, pharmaceutical composition comprises about 3.5% weight by volume benzyl alcohol. In some embodiments, pharmaceutical composition comprises about 4.0% weight by volume benzyl alcohol. In some embodiments, pharmaceutical composition comprises about 4.5% weight by volume benzyl alcohol. In some embodiments, pharmaceutical composition comprises about 5.0% weight by volume benzyl alcohol. In some embodiments, pharmaceutical composition comprises from about 1.0% to about 3.0% weight by volume benzyl alcohol.

In some embodiments, the pharmaceutical composition further comprises one or more of histidine, mannitol, propylene glycol, glycerin, arginine, lysine, tryptophan, or phenol. In some embodiments, the pharmaceutical composition further comprises histidine or tryptophan. In some embodiments, the pharmaceutical composition further comprises histidine. In some embodiments, the pharmaceutical composition further comprises mannitol. In some embodiments, the pharmaceutical composition further comprises propylene glycol. In some embodiments, the pharmaceutical composition further comprises glycerin. In some embodiments, the pharmaceutical composition further comprises arginine. In some embodiments, the pharmaceutical composition further comprises lysine. In some embodiments, the pharmaceutical composition further comprises tryptophan. In some embodiments, the pharmaceutical composition further comprises phenol.

In some embodiments, the pharmaceutical composition further comprises a buffer. Suitable buffers for administration of the compounds to a subject (e.g., via subcutaneous administration) are known in the art and within the purview of one skilled in the art. In some examples, the pharmaceutical composition further comprises a buffer selected from phosphate, histidine, or citrate. In some embodiments, the pharmaceutical composition further comprises histidine or citrate.

In some embodiments, the pharmaceutical composition further comprises histidine. In some embodiments, histidine is present in the pharmaceutical composition at a concentration ranging from about 5 mM to about 50 mM, more preferably from about 10 mM to about 40 mM, more preferably from about 15 mM to about 25 mM. In some embodiments, histidine is present in the pharmaceutical composition at a concentration of about 5 mM, about 10 mM, about 15 mM, about 20 mM, about 25 mM, about 30 mM, about 35 mM, about 40 mM, about 45 mM, or about 50 mM. In some embodiments, histidine is present in the pharmaceutical composition at a concentration from about 10 mM to about 30 mM. In some embodiments, histidine is present in the pharmaceutical composition at a concentration from about 15 mM to about 25 mM. In some embodiments, histidine is present in the pharmaceutical composition at a concentration of about 20 mM.

In some embodiments, the pharmaceutical composition further comprises citrate. In some embodiments, citrate is present in the pharmaceutical composition at a concentration ranging from about 5 mM to about 50 mM, more preferably from about 10 mM to about 40 mM, more preferably from about 15 mM to about 25 mM. In some embodiments, citrate is present in the pharmaceutical composition at a concentration of about 5 mM, about 10 mM, about 15 mM, about 20 mM, about 25 mM, about 30 mM, about 35 mM, about 40 mM, about 45 mM, or about 50 mM. In some embodiments, citrate is present in the pharmaceutical composition at a concentration from about 10 mM to about 30 mM. In some embodiments, citrate is present in the pharmaceutical composition at a concentration from about 15 mM to about 25 mM. In some embodiments, citrate is present in the pharmaceutical composition at a concentration of about 20 mM.

In some embodiments, the pharmaceutical composition further comprises tryptophan. In some embodiments, the tryptophan is present in a range from about 0.1% weight by volume to about 10% weight by volume, more preferably from about 0.5% weight by volume to about 7% weight by volume, more preferable from about 0.5% weight by volume to about 5% weight by volume. In some embodiments, the pharmaceutical composition comprises about 0.5% weight by volume tryptophan. In some embodiments, the pharmaceutical composition comprises about 1% weight by volume tryptophan. In some embodiments, the pharmaceutical composition comprises about 1.5% weight by volume tryptophan. In some embodiments, the pharmaceutical composition comprises about 2.0% weight by volume tryptophan. In some embodiments, the pharmaceutical composition comprises about 2.5% weight by volume tryptophan. In some embodiments, the pharmaceutical composition comprises about 3.0% weight by volume tryptophan. In some embodiments, the pharmaceutical composition comprises from about 0.5% to about 3% weight by volume tryptophan.

In some embodiments, the pharmaceutical composition further comprises propylene glycol. In some embodiments, the propylene glycol is present in a range from about 0.1% weight by volume to about 10% weight by volume, more preferably from about 0.5% weight by volume to about 7% weight by volume, more preferable from about 0.5% weight by volume to about 5% weight by volume. In some embodiments, the pharmaceutical composition comprises about 0.5% weight by volume propylene glycol. In some embodiments, the propylene glycol is present in a range from about 2% weight by volume to about 3% weight by volume. In some embodiments, the pharmaceutical composition comprises about 1% weight by volume propylene glycol. In some embodiments, pharmaceutical composition comprises about 1.5% weight by volume propylene glycol. In some embodiments, the pharmaceutical composition comprises about 2.0% weight by volume propylene glycol. In some embodiments, the pharmaceutical composition comprises about 2.1% weight by volume propylene glycol. In some embodiments, pharmaceutical composition comprises about 2.2% weight by volume propylene glycol. In some embodiments, the pharmaceutical composition comprises about 2.3% weight by volume propylene glycol. In some embodiments, the pharmaceutical composition comprises about 2.4% weight by volume propylene glycol. In some embodiments, the pharmaceutical composition comprises about 2.5% weight by volume propylene glycol. In some embodiments, the pharmaceutical composition comprises about 3.0% weight by volume propylene glycol. In some embodiments, the pharmaceutical composition comprises about 3.5% weight by volume propylene glycol. In some embodiments, the pharmaceutical composition comprises about 4.0% weight by volume propylene glycol. In some embodiments, the pharmaceutical composition comprises about 4.5% weight by volume propylene glycol. In some embodiments, the pharmaceutical composition comprises about 5.0% weight by volume propylene glycol. In some embodiments, the pharmaceutical composition comprises from about 1.0% to about 3.0% weight by volume propylene glycol.

In some embodiments, the pharmaceutical composition further comprises mannitol. In some embodiments, the mannitol is present in a range from about 0.1% weight by volume to about 10% weight by volume, more preferably from about 0.5% weight by volume to about 7% weight by volume, more preferable from about 1% weight by volume to about 7% weight by volume. In some embodiments, the pharmaceutical composition comprises about 1% weight by volume mannitol. In some embodiments, the pharmaceutical composition comprises about 2% weight by volume mannitol. In some embodiments, the pharmaceutical composition comprises about 3% weight by volume mannitol. In some embodiments, the pharmaceutical composition comprises about 4% weight by volume mannitol. In some embodiments, the pharmaceutical composition comprises about 4.5% weight by volume mannitol. In some embodiments, the pharmaceutical composition comprises about 5% weight by volume mannitol. In some embodiments, the pharmaceutical composition comprises about 5.5% weight by volume mannitol. In some embodiments, the pharmaceutical composition comprises about 6% weight by volume mannitol. In some embodiments, the pharmaceutical composition comprises about 7% weight by volume mannitol. In some embodiments, the pharmaceutical composition comprises about 8% weight by volume mannitol. In some embodiments, the pharmaceutical composition comprises from about 2.0% to about 8.0% weight by volume mannitol.

In some embodiments, the pharmaceutical composition further comprises histidine and propylene glycol.

In some embodiments, the pharmaceutical composition further comprises histidine and mannitol.

In some embodiments, the pharmaceutical composition has a pH in the range of about 5.5 to about 7.5. In some embodiments, the pharmaceutical composition has a pH in the range of about 6.0 to about 7.0. In some embodiments, the pharmaceutical composition has a pH in the range of about 6.5 to about 7.0. In some embodiments, the pharmaceutical composition has a pH of about 6.4. In some embodiments, the pharmaceutical composition has a pH of about 6.5. In some embodiments, the pharmaceutical composition has a pH of about 6.6. In some embodiments, the pharmaceutical composition has a pH of about 6.7. In some embodiments, the pharmaceutical composition has a pH of about 6.8. In some embodiments, the pharmaceutical composition has a pH of about 6.9. In some embodiments, the pharmaceutical composition has a pH of about 7.0. In some embodiments, the pharmaceutical composition has a pH of about 7.1.

In some embodiments, the pharmaceutical composition comprises from about 1.0% to about 3.0% weight by volume benzyl alcohol, and the composition has a pH from about 6.5 to about 7.0. In some embodiments, pharmaceutical composition comprises about 2.0% weight by volume benzyl alcohol, and the composition has a pH of about 6.5.

In some embodiments, the pharmaceutical composition comprises an antisense oligomer conjugate of the present disclosure, benzyl alcohol, histidine, and propylene glycol. In some embodiments, the pharmaceutical composition comprises from about 1.0% to about 3.0% weight by volume benzyl alcohol and from about 1.0% to about 3.0% weight by volume propylene glycol. In some embodiments, the pharmaceutical composition comprises from about 1.0% to about 3.0% weight by volume benzyl alcohol, histidine at a concentration from about 10 mM to about 30 mM, and from about 1.0% to about 3.0% weight by volume propylene glycol. In some embodiments, the pharmaceutical composition comprises from about 2.0% to about 3.0% weight by volume benzyl alcohol, histidine at a concentration from about 10 mM to about 30 mM, and from about 1.0% to about 3.0% weight by volume propylene glycol. In some embodiments, the pharmaceutical composition comprises about 2.0% weight by volume benzyl alcohol and about 2.0% weight by volume propylene glycol. In some embodiments, the pharmaceutical composition comprises about 2.0% weight by volume benzyl alcohol and about 2.2% weight by volume propylene glycol. In some embodiments, the pharmaceutical composition comprises about 2.0% weight by volume benzyl alcohol, histidine at a concentration of about 20 mM, and about 2.0% weight by volume propylene glycol. In some embodiments, the pharmaceutical composition has a pH of about 6.5.

In some embodiments, the pharmaceutical composition comprises an antisense oligomer conjugate of the present disclosure, benzyl alcohol, histidine, and mannitol. In some embodiments, the pharmaceutical composition comprises from about 1.0% to about 3.0% weight by volume benzyl alcohol and from about 2.0% to about 8.0% weight by volume mannitol. In some embodiments, the comprises from about 1.0% to about 3.0% weight by volume benzyl alcohol, histidine at a concentration from about 10 mM to about 30 mM, and from about 2.0% to about 8.0% weight by volume mannitol. In some embodiments, the pharmaceutical composition comprises about 2.0% weight by volume benzyl alcohol and about 5.0% weight by volume mannitol. In some embodiments, the pharmaceutical composition comprises about 2.0% weight by volume benzyl alcohol, histidine at a concentration of about 20 mM, and about 5.0% weight by volume mannitol. In some embodiments, the pharmaceutical composition has a pH of about 6.5.

In an embodiment, the pharmaceutical composition comprises about 2% weight by volume of benzyl alcohol and the composition has a pH of about 6.5. In another embodiment, the pharmaceutical composition comprises about 2% weight by volume of benzyl alcohol, about 2-3% weight by volume of propylene glycol, and the composition has a pH of about 6.5. In another embodiment, the pharmaceutical composition comprises about 2% weight by volume of benzyl alcohol, about 2.2% weight by volume of propylene glycol, and the composition has a pH of about 6.5. In another embodiment, the pharmaceutical composition comprises about 2% weight by volume of benzyl alcohol, about 5% weight by volume of mannitol, and the composition has a pH of about 6.5.

In some embodiments, the pharmaceutical composition has a viscosity of about 20 centipoise (cp) or less. In some embodiments, the pharmaceutical composition has a viscosity of about 10 cp or less. In some embodiments, the pharmaceutical composition has a viscosity of about 5 cp or less. In some embodiments, the pharmaceutical composition has a viscosity between about 2 cp and about 4 cp. In some embodiments, the pharmaceutical composition has a viscosity between about 2.5 cp and about 3.5 cp. In some embodiments, the pharmaceutical composition has a viscosity of about 3 cp.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

The pharmaceutical compositions of the present disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the antisense oligomer conjugate of formula (I) and benzyl alcohol of the instant disclosure include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The present disclosure also provides a composition comprising surface-modified liposomes containing poly (ethylene glycol) lipids (PEG-modified, branched and unbranched or combinations thereof, or long-circulating liposomes or stealth liposomes). Oligomers of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., PCT Publication No. WO 96/10391; Ansell et al., PCT Publication No. WO 96/10390; Holland et al., PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the compositions of the present disclosure include oligomer compositions prepared for delivery as described in U.S. Pat. Nos. 6,692,911, 7,163,695 and 7,070,807. In this regard, in one embodiment, the present disclosure provides an oligomer of the present invention in a composition comprising copolymers of lysine and histidine (HK) as described in U.S. Pat. Nos. 7,163,695, 7,070,807, and 6,692,911 either alone or in combination with PEG (e.g., branched or unbranched PEG or a mixture of both), in combination with PEG and a targeting moiety or any of the foregoing in combination with a crosslinking agent. In certain embodiments, the present disclosure provides compositions comprising an antisense oligomer conjugate, benzyl alcohol, and gluconic-acid-modified polyhistidine or gluconylated-polyhistidine/transferrin-polylysine. One skilled in the art will also recognize that amino acids with properties similar to His and Lys may be substituted within the composition.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In certain embodiments, a compositions of the present disclosure comprises an excipient selected from cyclodextrins, celluloses, liposomes, micelle forming agents, e.g., bile acids, and polymeric carriers, e.g., polyesters and polyanhydrides; and an antisense oligomer conjugate of formula (I); and benzyl alcohol. In certain embodiments, an aforementioned formulation renders orally bioavailable an oligomer of the present invention.

Compositions of the present disclosure include those suitable for oral, nasal, topical (including buccal and sublingual), rectal, vaginal and/or parenteral administration. In particular embodiments, the pharmaceutical composition is formulated for subcutaneous administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Methods of preparing these compositions include the step of bringing into association an antisense oligomer conjugate of formula (I), benzyl alcohol, and, optionally, one or more accessory ingredients or carriers. In general, the formulations are prepared by uniformly and intimately bringing into association a conjugate of the present disclosure with liquid carriers, or finely divided solid carriers, or both, and then, if necessary, shaping the product.

### Methods of Use and Additional Formulation Components

The present disclosure further provides methods for treating progeroid diseases, such as laminopathies and related diseases or conditions in a subject in need thereof by administering to the subject a pharmaceutical composition comprising benzyl alcohol and an antisense oligomer conjugate of formula (I) as described herein. In some embodiments, the oligonucleotide inhibits expression of mutant LMNA protein mRNA by modulating splicing of LMNA pre-mRNA. In a particular aspect, the present disclosure provides a method for treating Hutchinson-Gilford progeria syndrome (HGPS) in a subject in need thereof comprising administering to the subject a pharmaceutical composition of the present disclosure. In some embodiments, the pharmaceutical composition is formulated for subcutaneous administration to a subject. In some embodiments, the pharmaceutical composition is administered to a subject by subcutaneous administration.

In certain aspects, for example, these and related methods can be applied to treating progeroid laminopathies in clinical settings where progerin expression is associated with a disease such as HGPS. These and related embodiments can also be combined with methods of treating or reducing progeroid laminopathies, by concurrently or sequentially carrying out the methods of the disclosure with the other treatment.

The methods described herein can be generalized to the aging process and related conditions and diseases, beyond progeroid laminopathies. This is because HGPS is in many respects closely connected to normal aging processes. HGPS continues to be recognized as a useful model of aging (Fossel, J. Pediatr Endocrinol Metab 13 Suppl 6:1477-1481, 2000). For instance, the connection to atherosclerosis is very strong, especially of the coronary arteries. In addition, alopecia in HGPS is similar to that seen in subjects with advanced age. Further, the prime cellular feature of HGPS, as described many years ago by Hayflick and others (Hayflick, N Engl J Med 295: 1302-1308, 1976) is early cellular senescence. The limited number of cell divisions in HGPS fibroblasts is similar to what is seen in fibroblasts derived from elderly individuals. That was further explored by research showing similarities in the gene expression patterns of HGPS fibroblasts and those derived from elderly persons, distinguishing them from fibroblasts derived from younger persons (Ly et al., Science 287: 2486-2492, 2000).

Accordingly, it will be understood that a method for treating a progeroid disease or related condition as described herein can include the treatment of a progeroid laminopathy, such as HGPS, or another progeroid disease or condition, an age-related condition, a cardiovascular disease or condition (such as atherosclerosis), and the like.

It will be understood that an effective in vivo treatment regimen using the methods of the present disclosure may vary according to the duration, dose, frequency and route of administration of the pharmaceutical composition, as well as the condition of the subject under treatment (i.e., prophylactic administration versus administration in response to an existing condition). Accordingly, such in vivo therapy will often require monitoring by tests appropriate to the particular type of disease under treatment, and corresponding adjustments in the dose or treatment regimen, in order to achieve an optimal therapeutic outcome.

In certain embodiments, the methods of the present disclosure employ formulations or compositions suitable for the therapeutic delivery of antisense oligomers, as described herein. Hence, in certain embodiments, the methods of the present disclosure employ pharmaceutically acceptable compositions that comprise a therapeutically-effective amount of one or more of the antisense oligomer conjugates described herein, formulated together with benzyl alcohol, and optionally one or more pharmaceutically acceptable carriers (additives) and/or diluents.

Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT Application Publication No. WO 94/02595.

Compositions and formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A composition of the present disclosure may also be administered as a bolus, electuary or paste.

In solid dosage forms for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions used according to the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions or formulations for rectal or vaginal administration may be presented as a suppository, which may be prepared by mixing one or more conjugates of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Compositions or formulations for the topical or transdermal administration of a conjugate as provided herein include powders, sprays, ointments, pastes, creams, lotions, gels, solutions, patches and inhalants. The active oligomers may be mixed under sterile conditions with a pharmaceutically-acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a conjugate of the present disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a conjugate of the present disclosure to the body. Such dosage forms can be made by dissolving or dispersing the conjugate in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more antisense oligomer conjugate of formula (I) and benzyl alcohol in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions of the disclosure may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsuled matrices of the subject conjugates in biodegradable polymers such as polylactide-polyglycolide. Depending on the ratio of oligomer to polymer, and the nature of the particular polymer employed, the rate of oligomer release can be controlled. Examples of other biodegradable polymers include poly(orthoesters) and poly(anhydrides). Depot injectable formulations may also prepared by entrapping the drug in liposomes or microemulsions that are compatible with body tissues.

As noted above, the formulations or preparations used in the present disclosure may be given orally, parenterally, topically, or rectally. They are typically given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. In particular embodiments, the pharmaceutical composition is administered to a subject by subcutaneous administration.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutancous administration.

Regardless of the route of administration selected, the compositions of the present disclosure, may be formulated into pharmaceutically-acceptable dosage forms by conventional methods known to those of skill in the art. Actual dosage levels of the active ingredients in the pharmaceutical compositions of this invention may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular conjugate of the present disclosure employed, the route of administration, the time of administration, the rate of excretion or metabolism of the particular oligomer being employed, the rate and extent of absorption, the duration of the treatment, other drugs, compounds and/or materials used in combination with the particular oligomer employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the conjugates of the disclosure employed in the pharmaceutical composition at levels lower than that required in order to achieve the desired therapeutic effect and gradually increase the dosage until the desired effect is achieved. In general, a suitable daily dose of a conjugate of the disclosure will be that amount of the conjugate which is the lowest dose effective to produce a therapeutic effect. Such an effective dose will generally depend upon the factors described above. Generally, oral, intravenous, intracerebroventricular and subcutaneous doses of the conjugates of this disclosure for a patient, when used for the indicated effects, will range from about 0.0001 to about 100 mg per kilogram of body weight per day.

If desired, the effective daily dose of the conjugate may be administered as two, three, four, five, six or more sub-doses administered separately at appropriate intervals throughout the day, optionally, in unit dosage forms. In certain situations, dosing is one administration per day. In certain embodiments, dosing is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to treat the desired condition.

In certain embodiments, a formulation used in the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

An antisense oligomer conjugate may be formulated to be contained within, or, adapted to release by a surgical or medical device or implant. In certain aspects, an implant may be coated or otherwise treated with an oligomer. For example, hydrogels, or other polymers, such as biocompatible and/or biodegradable polymers, may be used to coat an implant with the compositions of the present invention (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

In addition to the methods provided herein, the compositions for use according to the present disclosure may be formulated for administration in any convenient way for use in human or veterinary medicine, by analogy with other pharmaceuticals. The compositions may be administered alone or in combination with other therapeutic strategies in the treatment of the relevant indication.

In accordance with the methods of the disclosure, routes of antisense oligomer delivery include, but are not limited to, various systemic routes, including oral and parenteral routes, e.g., intravenous, subcutaneous, intraperitoneal, and intramuscular, as well as inhalation, transdermal, pulmonary and topical delivery. The appropriate route may be determined by one of skill in the art, as appropriate to the condition of the subject under treatment. For example, an appropriate route for delivery of an antisense oligomer conjugate in the treatment of a condition of the skin may include topical delivery, while delivery of an antisense oligomer conjugate for the treatment of a respiratory condition (e.g., COPD) may include inhalation, intranasal or pulmonary delivery. The conjugate may also be delivered directly to a site of inflammation, infection, or to the bloodstream.

The composition may be administered in any convenient vehicle which is physiologically acceptable. Such a composition may include any of a variety of standard pharmaceutically acceptable carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration.

Sustained release compositions may also be used. These may include semipermeable polymeric matrices in the form of shaped articles such as films or microcapsules.

In certain embodiments, the compositions may be administered in an amount and manner effective to result in a peak blood concentration of at least 200-400 nM antisense oligomer conjugate. Typically, one or more doses of the conjugate are administered, generally at regular intervals, for a period of about one to two weeks. Preferred doses for oral administration are from about 1-100 mg conjugate per 70 kg. In some cases, doses of greater than 100 mg conjugate/patient may be necessary. For i.v. administration, preferred doses are from about 1 mg to 500 mg conjugate per 70 kg. The conjugate may be administered at regular intervals for a short time period, e.g., daily for two weeks or less. However, in some cases the conjugate is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

### Kits

In another aspect of the present disclosure, kits are provided. Kits according to the disclosure include package(s) comprising pharmaceutical compositions of the disclosure. In some embodiments, kits comprise benzyl alcohol and an antisense oligonucleotide conjugate according to Formula I, or a pharmaceutically acceptable salt thereof.

The phrase “package” means any vessel containing the pharmaceutical compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering the compositions of the disclosure to a patient. Kits also can comprise instructions for approved uses of the antisense oligonucleotide conjugates described herein by regulatory agencies, such as the United States Food and Drug Administration. Kits can also contain labeling or product inserts for the conjugates. The package(s) or any product insert(s), or both, may themselves be approved by regulatory agencies. The kits can also include buffers for preparing solutions for conducting the methods, and pipettes for transferring liquids from one container to another.

### EXAMPLES

Examples have been set forth below for the purpose of illustration and to describe certain specific embodiments of the disclosure. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations or methods of the disclosure may be made without departing from the spirit of the disclosure and the scope of the appended claims. Definitions of the variables in the structures in the schemes herein are commensurate with those of corresponding positions in the formulae presented herein.

### Example 1: Viscosity Study of an Antisense Oligomer Conjugate

To determine the highest allowable concentration of an antisense oligomer conjugate of the present disclosure for subcutaneous administration (viscosity≤ 20 cp), solutions at varying concentrations of the antisense oligomer conjugate of formula (IVb) were prepared and tested for viscosity as well as syringeability.

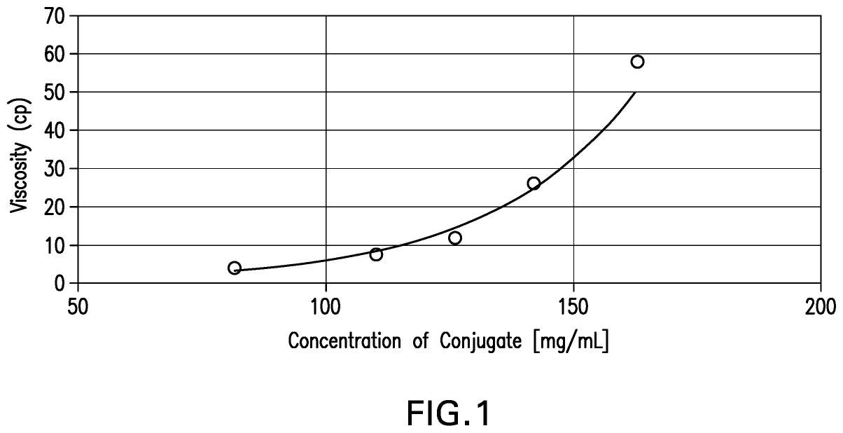

Test conditions were prepared from solid conjugate material in buffered solutions (50 mM citrate, pH=6.5), non-volumetrically. Viscosity was determined at room temperature, except for the samples at 50 mg/mL and 100 mg/mL of the conjugate, which were tested both at room temperature and after storage at 2-8° C. for 24 hours. No pH adjustments were made prior to Q.S. Syringeability was performed using a 30-gauge needle. Recovery and chromatographic overlays were determined via SCX-HPLC and SEC-HPLC, respectively. Results of the study are shown in Table 5 and FIG. 1.

TABLE 5
Results of viscosity testing

| Concentration of | | Viscosity | Syringe- | |
|---|---|---|---|---|
| Conjugate (mL) | | (cp) | ability | Appearance |
| 47.1 ± 0.5 | mg/mL | 1.8 (rt) | Yes | Clear; smooth, stream- |
| | | 1.9 (2-8° C.) | | like flow from needle |
| 81.6 ± 1.1 | mg/mL | 3.8 (rt) | Yes | Clear; smooth, stream- |
| | | 4.0 (2-8° C.) | | like flow from needle |
| 110.2 ± 0.2 | mg/mL | 7.3 | Yes | Clear; smooth, stream- |
| | | | | like flow from needle |
| 126.1 ± 0.6 | mg/mL | 11.8 | Yes | Clear; smooth, stream- |
| | | | | like flow from needle |
| 142.0 ± 0.8 | mg/mL | 26.1 | Yes | Clear; slow draw; |
| | | | | flows as droplets from |
| | | | | needle; increased back |
| | | | | pressure |
| 162.9 ± 2.5 | mg/mL | 57.8 | No | Clear; too viscous to |
| | | | | load needle |
| 169.5 ± 3.6 | mg/mL | 447.2 | No | Clear; too viscous to |
| | | | | load needle |
| 253.3 ± 3.4 | mg/mL | >2000 | No | Clear; too viscous to |
| | | | | load needle |

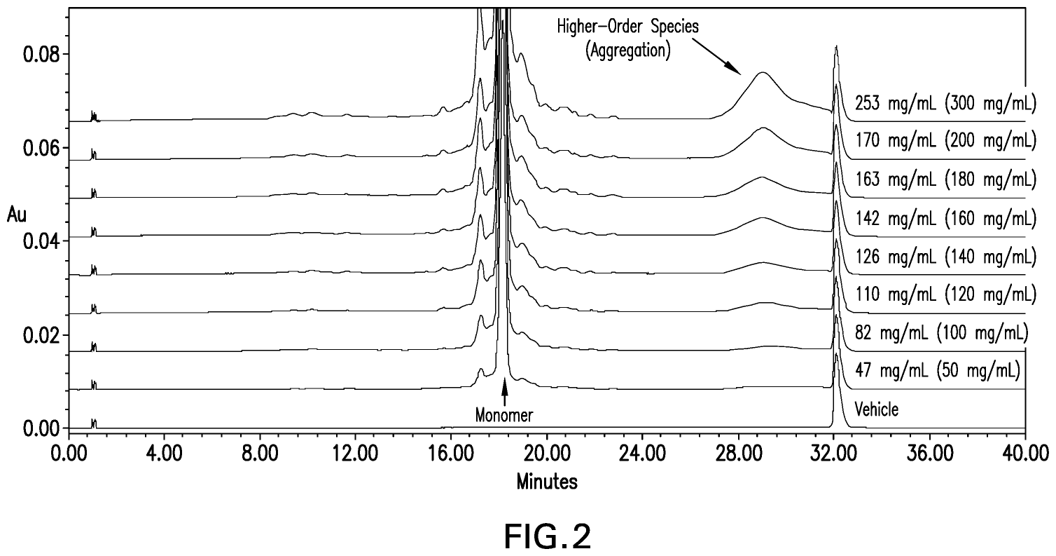

Viscosity of the tested antisense oligomer conjugate was found to increase exponentially at increasing concentrations. Concentrations below 126 mg/mL (˜12 cp) were found to be syringeable through a 30-cc gauge needle. A small increase in viscosity (<1 cP) was reported after overnight storage at 2-8° C. The presence of higher-order species (as a result of aggregation) was observed in all test solutions and was found to be concentration-dependent (FIG. 2).

### Example 2: Excipient Studies

Several excipients, compatible for subcutaneous administration, were screened with the objective of reducing the formation of higher-order species that might reduce the concentration of the antisense oligomer conjugate upon administration as well as display potential toxicity.

### Excipient Screening: Round 1

Formulations were prepared and screened to determine the effect that buffer type (phosphate vs. citrate) plays on the suppression of higher-order species (aggregation). The effect of various excipient types (e.g., amino acids, cosolvents, salts, and sugar alcohols) on aggregation was also investigated.

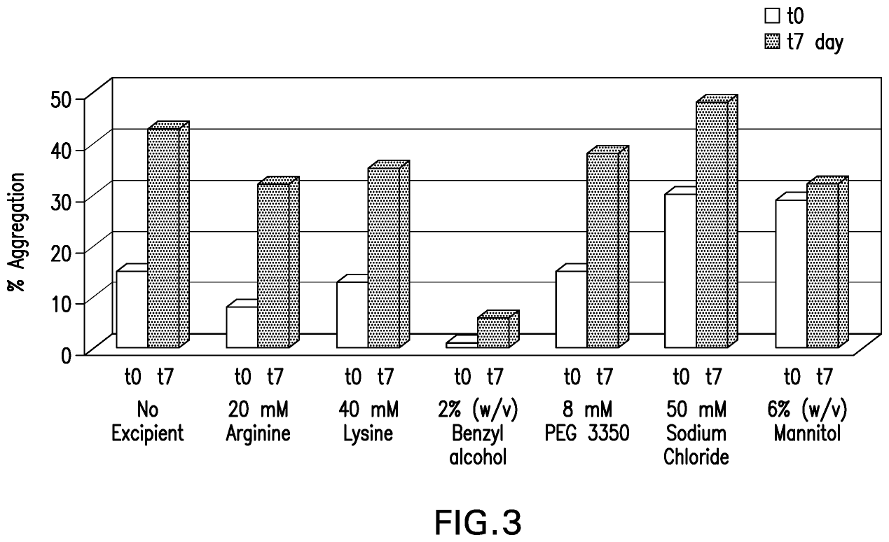

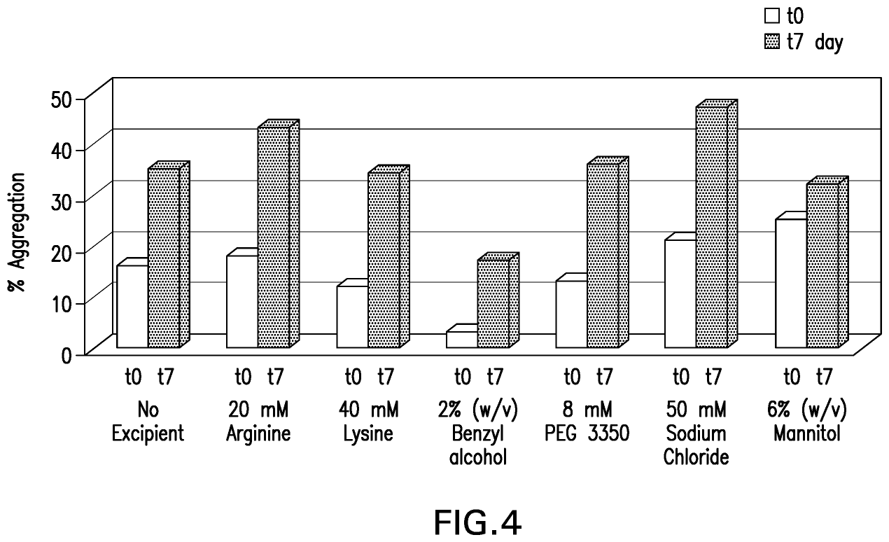

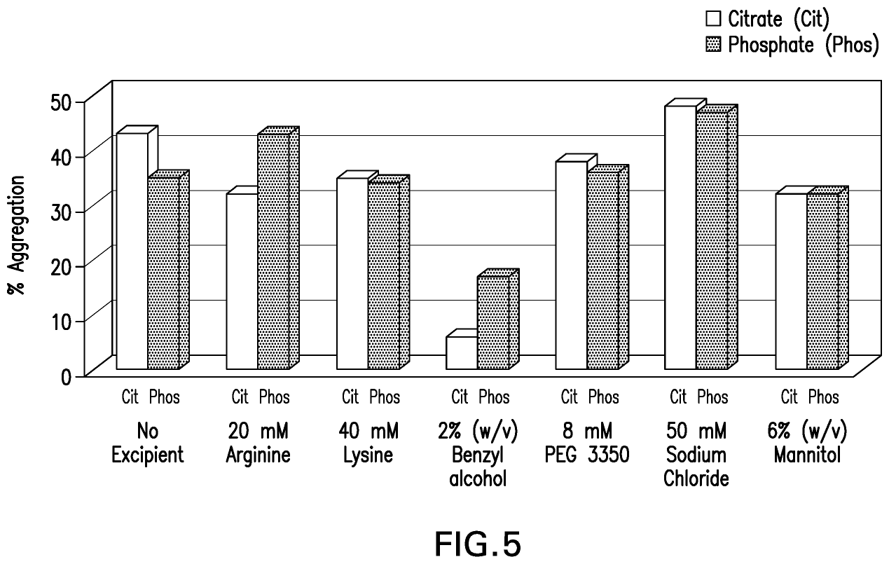

Test solutions were prepared at 100 mg/mL of the antisense oligomer conjugate of formula (IVb) from solid material and excipients in volumetric flasks with 20 mM citrate or potassium phosphate buffer (both at pH=6.5). The samples were adjusted to a pH of about 6.5 prior to Q.S. The samples were stored at either room temperature or 2-8° C. between time points, as indicated in the tables below, and analyzed via SEC-HPLC. Results of the study are shown in Tables 6 and 7 and in FIGS. 3, 4, and 5.

TABLE 6
Results of excipient screening in citrate buffer

| | | % Higher | | | | | |
|---|---|---|---|---|---|---|---|
| | | Order | | | | | |
| | Time | Species | % | Osmolarity | Viscosity | Syringe- | |
| Excipient | Point | (Aggregation) | Monomer | (mOsm/Kg) | (cp) | ability | Appearance |
| none | t₀ (rt) | 15 | 85 | 118 | 3.0 | — | Clear |
| | t₇ day | 43 | 57 | | | | |
| | (2- | | | | | | |
| | 8° C.) | | | | | | |
| 20 mM | t₀ (rt) | 8 | 92 | 117 | 2.6 | Yes | Clear; |
| Arginine | t₇ day | 32 | 68 | | | | smooth, |
| | (2- | | | | | | stream-like |
| | 8° C.) | | | | | | flow from |
| | | | | | | | needle |
| 40 mM | t₀ (rt) | 13 | 87 | 143 | 3.2 | Yes | Clear; |
| Lysine | t₇ day | 35 | 65 | | | | smooth, |
| | (2- | | | | | | stream-like |
| | 8° C.) | | | | | | flow from |
| | | | | | | | needle |
| 2% (w/v) | t₀ (rt) | 1 | 99 | 113 | 2.5 | Yes | Clear; |
| Benzyl | t7 day | 6 | 94 | | | | smooth, |
| alcohol | (2- | | | | | | stream-like |
| | 8° C.) | | | | | | flow from |
| | | | | | | | needle |
| 8 mM | t₀ (rt) | 15 | 85 | 131 | 4.5 | Yes | Clear; |
| PEG | t₇ day | 38 | 62 | | | | smooth, |
| 3350 | (2- | | | | | | stream-like |
| | 8° C.) | | | | | | flow from |
| | | | | | | | needle |
| 50 mM | t₀ (rt) | 30 | 70 | 200 | 2.7 | Yes | Clear; |
| NaCl | t₇ day | 48 | 52 | | | | smooth, |
| | (2- | | | | | | stream-like |
| | 8° C.) | | | | | | flow from |
| | | | | | | | needle |
| 6% (w/v) | t₀ (rt) | 29 | 71 | 527 | 3.5 | Yes | Clear; |
| Mannitol | t₇ day | 32 | 68 | | | | smooth, |
| | (2- | | | | | | stream-like |
| | 8° C.) | | | | | | flow from |
| | | | | | | | needle |

TABLE 7
Results of excipient screening in phosphate buffer

| | | % Higher | | | | | |
|---|---|---|---|---|---|---|---|
| | | Order | | | | | |
| | Time | Species | % | Osmolarity | Viscosity | Syringe- | |
| Excipient | Point | (Aggregation) | Monomer | (mOsm/Kg) | (cp) | ability | Appearance |
| none | t₀ (rt) | 16 | 84 | 86 | 3.8 | — | Clear |
| | t₇ day | 35 | 65 | | | | |
| | (2- | | | | | | |
| | 8° C.) | | | | | | |
| 20 mM | t₀ (rt) | 18 | 82 | 109 | 3.4 | Yes | Clear; |
| Arginine | t₇ day | 43 | 57 | | | | smooth, |
| | (2- | | | | | | stream-like |
| | 8° C.) | | | | | | flow from |
| | | | | | | | needle |
| 40 mM | t₀ (rt) | 12 | 88 | 109 | 2.9 | Yes | Clear; |
| Lysine | t₇ day | 34 | 66 | | | | smooth, |
| | (2- | | | | | | stream-like |
| | 8° C.) | | | | | | flow from |
| | | | | | | | needle |
| 2% (w/v) | t₀ (rt) | 3 | 97 | 73 | 2.8 | Yes | Clear; |
| Benzyl | t₇ day | 17 | 83 | | | | smooth, |
| alcohol | (2- | | | | | | stream-like |
| | 8° C.) | | | | | | flow from |
| | | | | | | | needle |
| 8 mM | t₀ (rt) | 13 | 87 | 114 | 3.9 | Yes | Clear; |
| PEG | t₇ day | 36 | 64 | | | | smooth, |
| 3350 | (2- | | | | | | stream-like |
| | 8° C.) | | | | | | flow from |
| | | | | | | | needle |
| 50 mM | t₀ (rt) | 21 | 79 | 168 | 3.0 | Yes | Clear; |
| NaCl | t₇ day | 47 | 53 | | | | smooth, |
| | (2- | | | | | | stream-like |
| | 8° C.) | | | | | | flow from |
| | | | | | | | needle |
| 6% (w/v) | t₀ (rt) | 25 | 75 | 484 | 3.4 | Yes | Clear; |
| Mannitol | t₇ day | 32 | 68 | | | | smooth, |
| | (2- | | | | | | stream-like |
| | 8° C.) | | | | | | flow from |
| | | | | | | | needle |

Of the excipients tested, benzyl alcohol had the greatest protective effect against the formation of higher order species in either citrate or phosphate buffer. Arginine was the second best-performing excipient when tested in citrate buffer, though no effects were observed for arginine in phosphate buffer. Of the two buffers tested, citrate appeared to confer a protective effect, particularly for the benzyl alcohol and arginine compositions. Most samples were found to be hypotonic.

### Excipient Screening: Round 2

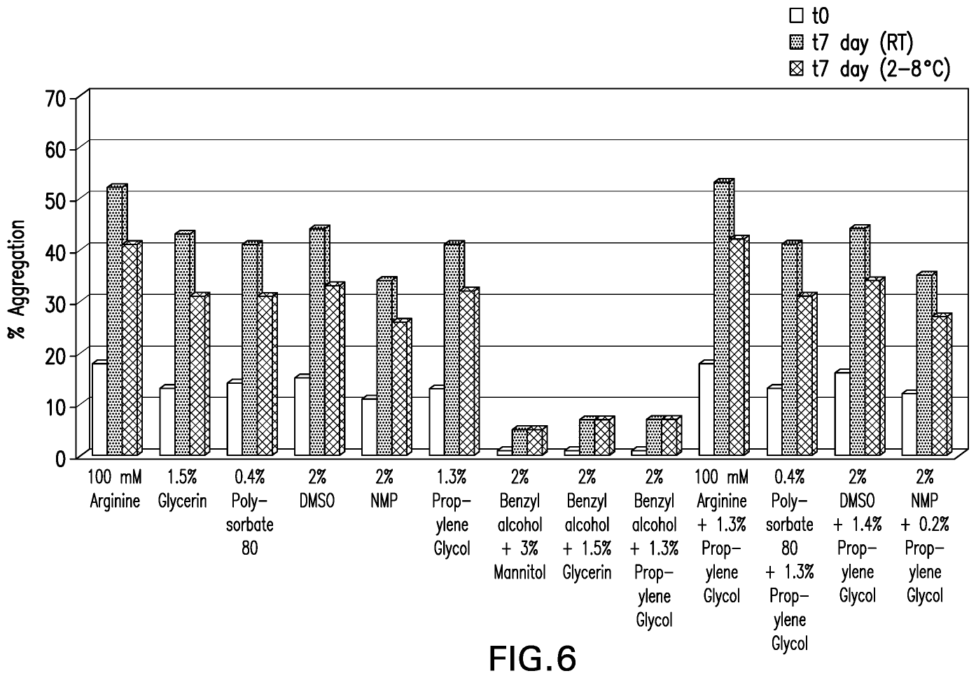

A second round of screening was performed to explore the excipients chemical space of the best performing conditions from Round 1 as well as to optimize the formulation osmolarity by use of sugars and poly-alcohols. Formulations were prepared and tested as described for Round 1. Results of the study are shown in Table 8 and in FIG. 6.

TABLE 8
Results of excipient screening

| | | % Higher | | | | | |
|---|---|---|---|---|---|---|---|
| | Time | Order Species | % | Osmolarity | Viscosity | Syringe- | |
| Excipient(s) | Point | (Aggregation) | Monomer | (mOsm/Kg) | (cp) | ability | Appearance |
| Arginine | t₀ (rt) | 18 | 82 | 388 | 4.0 | Yes | Clear; smooth, |
| (100.6 mM) | t₇ day | 52 | 48 | | | | stream-like |
| | (rt) | | | | | | flow from |
| | t₇ day | 41 | 59 | | | | needle |
| | (2-8° C.) | | | | | | |
| Glycerin | t₀ (rt) | 13 | 87 | 316 | 3.1 | Yes | Clear; smooth, |
| (1.5%) | t₇ day | 43 | 57 | | | | stream-like |
| | (rt) | | | | | | flow from |
| | t₇ day | 31 | 69 | | | | needle |
| | (2-8° C.) | | | | | | |
| Polysorbate | t₀ (rt) | 14 | 86 | 126 | 3.2 | Yes | Clear; smooth, |
| 80 (0.4%) | t₇ day | 42 | 58 | | | | stream-like |
| | (rt) | | | | | | flow from |
| | t₇ day | 31 | 69 | | | | needle; |
| | (2-8° C.) | | | | | | slightly foamy |
| DMSO | t₀ (rt) | 15 | 85 | 379 | 3.0 | Yes | Clear; smooth, |
| (2.0%) | t₇ day | 44 | 56 | | | | stream-like |
| | (rt) | | | | | | flow from |
| | t₇ day | 33 | 67 | | | | needle |
| | (2-8° C.) | | | | | | |
| NMP | t₀ (rt) | 11 | 89 | 280 | 2.9 | Yes | Clear; smooth, |
| (2.0%) | t₇ day | 34 | 66 | | | | stream-like |
| | (rt) | | | | | | flow from |
| | t₇ day | 26 | 74 | | | | needle |
| | (2-8° C.) | | | | | | |
| Propylene | t₀ (rt) | 13 | 87 | 281 | 3.1 | Yes | Clear; smooth, |
| glycol | t₇ day | 41 | 59 | | | | stream-like |
| (1.3%) | (rt) | | | | | | flow from |
| | t₇ day | 32 | 68 | | | | needle |
| | (2-8° C.) | | | | | | |
| Benzyl | t₀ (rt) | 1 | 99 | 313 | 3.0 | Yes | Clear; smooth, |
| alcohol | t₇ day | 5 | 95 | | | | stream-like |
| (2.0%) | (rt) | | | | | | flow from |
| + | t₇ day | 5 | 95 | | | | needle |
| Mannitol | (2-8° C.) | | | | | | |
| (3.0%) | | | | | | | |
| Benzyl | t₀ (rt) | 1 | 99 | 283 | 2.9 | Yes | Clear; smooth, |
| alcohol | t₇ day | 7 | 93 | | | | stream-like |
| (2.0%) | (rt) | | | | | | flow from |
| + | t₇ day | 7 | 93 | | | | needle |
| Propylene | (2-8° C.) | | | | | | |
| glycol | | | | | | | |
| (1.3%) | | | | | | | |
| Benzyl | t₀ (rt) | 1 | 99 | 313 | 2.9 | Yes | Clear; smooth, |
| alcohol | t₇ day | 7 | 93 | | | | stream-like |
| (2.0%) | (rt) | | | | | | flow from |
| + | t₇ day | 7 | 93 | | | | needle |
| Glycerin | (2-8° C.) | | | | | | |
| (1.5%) | | | | | | | |
| Arginine | t₀ (rt) | 18 | 82 | 537 | 3.5 | Yes | Clear; smooth, |
| (100.6 mM) | t₇ day | 53 | 47 | | | | stream-like |
| + | (rt) | | | | | | flow from |
| Propylene | t₇ day | 42 | 58 | | | | needle |
| glycol | (2-8° C.) | | | | | | |
| (1.3%) | | | | | | | |
| Polysorbate | t₀ (rt) | 13 | 87 | 297 | 3.1 | Yes | Clear; smooth, |
| 80 (0.4%) | t₇ day | 41 | 59 | | | | stream-like |
| + | (rt) | | | | | | flow from |
| Propylene | t₇ day | 31 | 69 | | | | needle; |
| glycol | (2-8° C.) | | | | | | slightly foamy |
| (1.3%) | | | | | | | |
| DMSO | t₀ (rt) | 16 | 84 | 569 | 3.1 | Yes | Clear; smooth, |
| (2.0%) | t₇ day | 44 | 56 | | | | stream-like |
| + | (rt) | | | | | | flow from |
| Propylene | t₇ day | 34 | 66 | | | | needle |
| glycol | (2-8° C.) | | | | | | |
| (1.3%) | | | | | | | |
| NMP | t₀ (rt) | 12 | 88 | 301 | 3.0 | Yes | Clear; smooth, |
| (2.0%) | t₇ day | 35 | 65 | | | | stream-like |
| + | (rt) | | | | | | flow from |
| Propylene | t₇ day | 27 | 73 | | | | needle |
| glycol | (2-8° C.) | | | | | | |
| (1.3%) | | | | | | | |

Testing of additional co-solvents did not appear to significantly affect aggregation. Of the excipients and excipient combinations tested, the benzyl alcohol compositions were the only ones capable of minimizing aggregation of the antisense oligonucleotide conjugate. Small decreases in aggregation were reported in the presence of glycerin, NMP, and propylene glycol only upon preparation (<5%). Generally, samples stored at room temperature were observed to have higher aggregation than 2-8° C. samples, likely due to increased molecular mobility. Most formulations were reported to be isotonic.

### Excipient Screening: Round 3

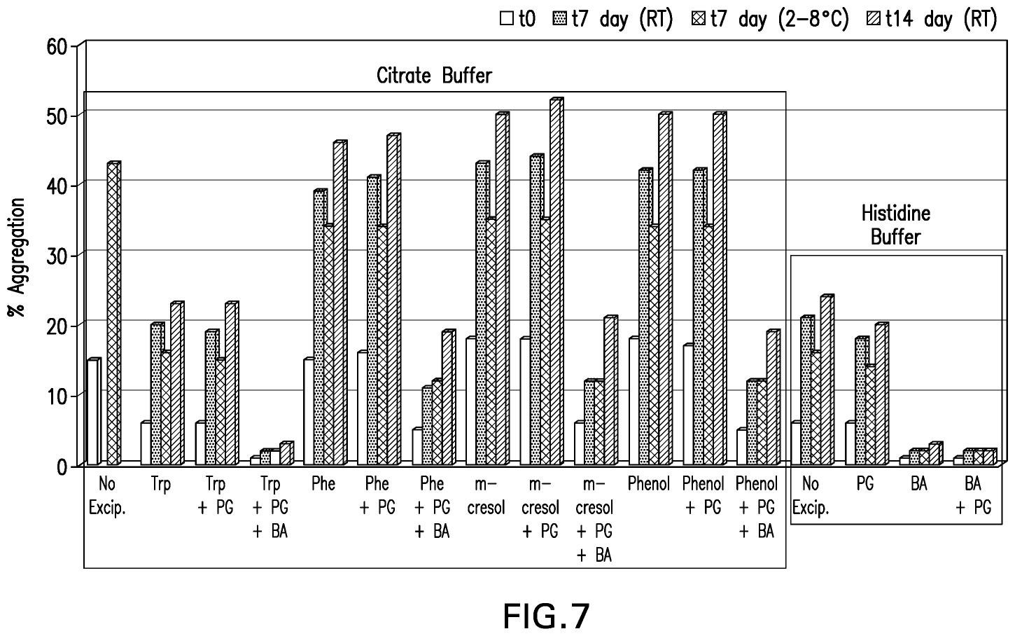

A third round of screening as performed to evaluate alternate aromatic excipients compatible for subcutaneous administration, noting that protective effect of benzyl alcohol could be due to pi-stacking interactions occurring between the solvent and the antisense oligonucleotide conjugate. Solutions were prepared and tested as described in Round 1. Results of the study are shown in Table 9 and in FIG. 7.

TABLE 9
Results of excipient screening

| | | % Higher | | | | | |
|---|---|---|---|---|---|---|---|
| | Time | Order Species | % | Osmolarity | Viscosity | Syringe- | |
| Excipient(s) | Point | (Aggregation) | Monomer | (mOsm/Kg) | (cp) | ability | Appearance |
| Tryptophan | t₀ (rt) | 6 | 94 | 145 | 3.0 | Yes | Clear; |
| (1%) | t₇ day | 20 | 80 | | | | smooth, |
| | (rt) | | | | | | stream-like |
| | t₇ day | 16 | 84 | | | | flow from |
| | (2-8° C.) | | | | | | needle |
| | t₁₄ day | 23 | 77 | | | | |
| | (rt) | | | | | | |
| Tryptophan | t₀ (rt) | 6 | 94 | 312 | 3.1 | Yes | Clear; |
| (1%) | t₇ day | 19 | 81 | | | | smooth, |
| + | (rt) | | | | | | stream-like |
| Propylene | t₇ day | 15 | 85 | | | | flow from |
| glycol (1%) | (2-8° C.) | | | | | | needle |
| | t₁₄ day | 23 | 77 | | | | |
| | (rt) | | | | | | |
| Tryptophan | t₀ (rt) | 1 | 99 | 307 | 3.0 | Yes | Clear; |
| (1%) | t₇ day | 2 | 98 | | | | smooth, |
| + | (rt) | | | | | | stream-like |
| Propylene | t₇ day | 2 | 98 | | | | flow from |
| glycol (1%) | (2-8° C.) | | | | | | needle |
| + | t₁₄ day | 3 | 97 | | | | |
| Benzyl | (rt) | | | | | | |
| alcohol (2%) | | | | | | | |
| Phenylalanine | t₀ (rt) | 15 | 85 | 174 | 3.3 | Yes | Clear; |
| (1%) | t₇ day | 39 | 61 | | | | smooth, |
| | (rt) | | | | | | stream-like |
| | t₇ day | 34 | 66 | | | | flow from |
| | (2-8° C.) | | | | | | needle |
| | t₁₄ day | 46 | 54 | | | | |
| | (rt) | | | | | | |
| Phenylalanine | t₀ (rt) | 16 | 84 | 322 | 3.0 | Yes | Clear; |
| (1%) | t₇ day | 41 | 59 | | | | smooth, |
| + | (rt) | | | | | | stream-like |
| Propylene | t₇ day | 34 | 66 | | | | flow from |
| glycol (1%) | (2-8° C.) | | | | | | needle |
| | t₁₄ day | 47 | 53 | | | | |
| | (rt) | | | | | | |
| Phenylalanine | t₀ (rt) | 5 | 95 | 316 | 3.1 | Yes | Clear; |
| (1%) | t₇ day | 11 | 89 | | | | smooth, |
| + | (rt) | | | | | | stream-like |
| Propylene | t₇ day | 12 | 88 | | | | flow from |
| glycol (1%) | (2-8° C.) | | | | | | needle |
| + | t₁₄ day | 19 | 81 | | | | |
| Benzyl | (rt) | | | | | | |
| alcohol (2%) | | | | | | | |
| m-Cresol | t₀ (rt) | 18 | 82 | 227 | 3.2 | Yes | Clear; |
| (0.3%) | t₇ day | 43 | 57 | | | | smooth, |
| | (rt) | | | | | | stream-like |
| | t₇ day | 35 | 65 | | | | flow from |
| | (2-8° C.) | | | | | | needle |
| | t₁₄ day | 50 | 50 | | | | |
| | (rt) | | | | | | |
| m-Cresol | t₀ (rt) | 18 | 82 | 282 | 3.2 | Yes | Clear; |
| (0.3%) | t₇ day | 44 | 56 | | | | smooth, |
| + | (rt) | | | | | | stream-like |
| Propylene | t₇ day | 35 | 65 | | | | flow from |
| glycol (2%) | (2-8° C.) | | | | | | needle |
| | t₁₄ day | 52 | 48 | | | | |
| | (rt) | | | | | | |
| m-Cresol | t₀ (rt) | 6 | 94 | 277 | 3.2 | Yes | Clear; |
| (0.3%) | t₇ day | 12 | 88 | | | | smooth, |
| + | (rt) | | | | | | stream-like |
| Propylene | t₇ day | 12 | 88 | | | | flow from |
| glycol (1%) | (2-8° C.) | | | | | | needle |
| + | t₁₄ day | 21 | 79 | | | | |
| Benzyl | (rt) | | | | | | |
| alcohol (2%) | | | | | | | |
| Phenol | t₀ (rt) | 18 | 82 | 117 | 3.1 | Yes | Clear; |
| (0.5%) | t₇ day | 42 | 58 | | | | smooth, |
| | (rt) | | | | | | stream-like |
| | t₇ day | 34 | 66 | | | | flow from |
| | (2-8° C.) | | | | | | needle |
| | t₁₄ day | 50 | 50 | | | | |
| | (rt) | | | | | | |
| Phenol | t₀ (rt) | 17 | 83 | 291 | 3.1 | Yes | Clear; |
| (0.5%) | t₇ day | 42 | 58 | | | | smooth, |
| + | (rt) | | | | | | stream-like |
| Propylene | t₇ day | 34 | 66 | | | | flow from |
| glycol (2%) | (2-8° C.) | | | | | | needle; |
| | t₁₄ day | 50 | 50 | | | | slightly foamy |
| | (rt) | | | | | | |
| Phenol | t₀ (rt) | 5 | 95 | 286 | 3.1 | Yes | Clear; |
| (0.5%) | t₇ day | 12 | 88 | | | | smooth, |
| + | (rt) | | | | | | stream-like |
| Propylene | t₇ day | 12 | 88 | | | | flow from |
| glycol (2%) | (2-8° C.) | | | | | | needle |
| + | t₁₄ day | 19 | 81 | | | | |
| Benzyl | (rt) | | | | | | |
| alcohol (2%) | | | | | | | |
| Histidine | t₀ (rt) | 6 | 94 | 85 | 2.9 | Yes | Clear; |
| (20 mM) | t₇ day | 21 | 79 | | | | smooth, |
| | (rt) | | | | | | stream-like |
| | t₇ day | 16 | 84 | | | | flow from |
| | (2-8° C.) | | | | | | needle |
| | t₁₄ day | 24 | 76 | | | | |
| | (rt) | | | | | | |
| Histidine | t₀ (rt) | 6 | 94 | 312 | 3.1 | Yes | Clear; |
| (20 mM) | t₇ day | 18 | 82 | | | | smooth, |
| + | (rt) | | | | | | stream-like |
| Propylene | t₇ day | 14 | 86 | | | | flow from |
| glycol (2%) | (2-8° C.) | | | | | | needle |
| | t₁₄ day | 20 | 80 | | | | |
| | (rt) | | | | | | |
| Histidine | t₀ (rt) | 1 | 99 | 75 | 2.7 | Yes | Clear; |
| (20 mM) | t₇ day | 2 | 98 | | | | smooth, |
| + | (rt) | | | | | | stream-like |
| Benzyl | t₇ day | 2 | 98 | | | | flow from |
| alcohol (2%) | (2-8° C.) | | | | | | needle |
| | t₁₄ day | 3 | 97 | | | | |
| | (rt) | | | | | | |
| Histidine | t₀ (rt) | 1 | 99 | 303 | 2.7 | Yes | Clear; |
| (20 mM) | t₇ day | 2 | 98 | | | | smooth, |
| + | (rt) | | | | | | stream-like |
| Propylene | t₇ day | 2 | 98 | | | | flow from |
| glycol (2%) | (2-8° C.) | | | | | | needle |
| + | | | | | | | |
| Benzyl | t₁₄ day | 2 | 98 | | | | |
| alcohol (2%) | (rt) | | | | | | |

When used as a single excipient, tryptophan and histidine showed some reductions in conjugate aggregation (approximately 9%) upon preparation. Combinations with benzyl alcohol showed significant reduction in aggregation even after two weeks of storage at room temperature for both excipients. Without wishing to be bound to theory, it is believed that pi-stacking interactions between aromatic groups is still the main reason for the increased physical stability. No significant effects were reported for phenol, phenylalanine, and m-cresol when used as single excipients.

### Example 3: Accelerated Stability Studies

The stability of a composition comprising the antisense oligomer conjugate of formula (IVb) was determined under accelerated conditions. Vehicles were designed based on the results from the screening rounds described in Example 2 and cover a formulation space of two buffers (citrate and histidine), two tonicifiers (mannitol and propylene glycol), one cosolvent (benzyl alcohol), and one stabilizer (tryptophan) at pHs ranging from 6.0 to 7.0. The four formulations screened in the accelerated stability studies are described in Table 10.

TABLE 10
Formulations for accelerated stability studies

| Formulation A | Formulation B | Formulation C | Formulation D |
|---|---|---|---|
| Antisense oligomer | Antisense oligomer | Antisense oligomer | Antisense oligomer |
| conjugate (100 mg/mL) | conjugate (100 mg/mL) | conjugate (100 mg/mL) | conjugate (100 mg/mL) |
| Citrate (20 mM) | Citrate (20 mM) | Histidine (20 mM) | Histidine (20 mM) |
| Benzyl alcohol (2%) | Benzyl alcohol (2%) | Benzyl alcohol (2%) | Benzyl alcohol (2%) |
| Propylene glycol (1%) | Mannitol (3%) | Mannitol (5%) | Propylene glycol (2%) |
| Tryptophan (1%) | Tryptophan (1%) | Prepared at pH 6.0, | Prepared at pH 6.0, |
| Prepared at pH 6.0, | Prepared at pH 6.0, | 6.5, and 7.0 | 6.5, and 7.0 |
| 6.5, and 7.0 | 6.5, and 7.0 | | |

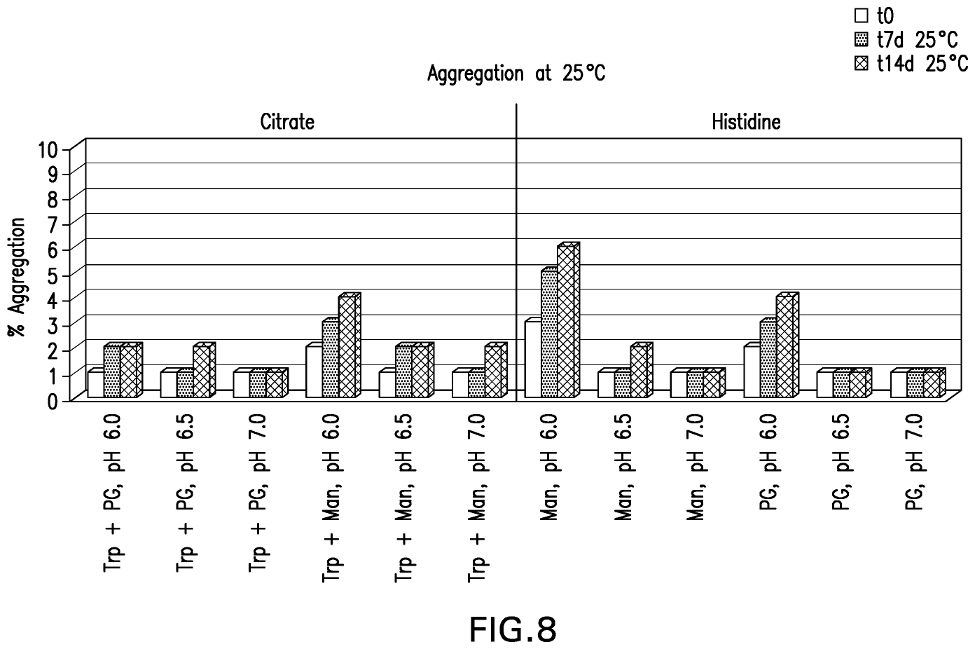

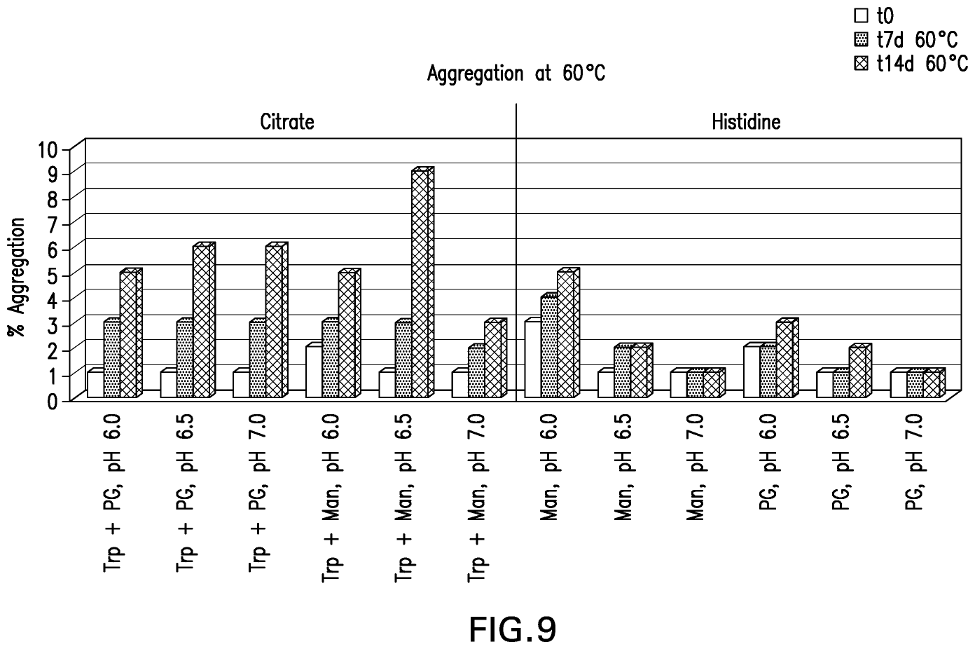

Formulations were prepared from solid PPMO material in vehicle, volumetrically. The pH of each formulation was adjusted to target prior to Q.S. The samples were placed under accelerated stability conditions of 60° C. and were tested via SCX-HPLC and SEC-HPLC upon preparation, after seven days, and after fourteen days. Recovery was set to 100% for the pre-filtered samples. Purity is based on the relative total peak area derived from SCX-HPLC analysis. Results of the study are shown in Tables 11-18 and in FIGS. 8 and 9.

TABLE 11
Aggregation analysis of Formulation A* at 60° C.

| | | % Higher | | | | | |
|---|---|---|---|---|---|---|---|
| | | Order | | | | | |
| Observed | Time | Species | % | Osmolarity | Viscosity | Syringe- | |
| pH | Point | (Aggregation) | Monomer | (mOsm/Kg) | (cp) | ability | Appearance |
| 6.0 | t₀ | 1 | 99 | 303 | 3.3 | Smooth | Clear |
| 6.0 | t₇ day | 3 | 97 | 311 | 3.5 | Smooth | Dark orange/ |
| | | | | | | | yellow |
| 6.0 | t₁₄ day | 5 | 95 | 317 | 3.8 | Smooth | Dark brown |
| 6.5 | t₀ | 1 | 99 | 302 | 3.3 | Smooth | Clear |
| 6.4 | t₇ day | 3 | 97 | 330 | 3.5 | Smooth | Dar orange/ |
| | | | | | | | yellow |
| 6.4 | t₁₄ day | 6 | 94 | 310 | 3.7 | Smooth | Dark brown |
| 7.0 | t₀ | 1 | 99 | 292 | 3.2 | Smooth | Clear |
| 6.6 | t₇ day | 3 | 97 | 303 | 3.4 | Smooth | Dark orange/ |
| | | | | | | | yellow |
| 6.6 | t₁₄ day | 6 | 94 | 307 | 3.7 | Smooth | Dark brown |

*citrate buffer (20 mM), benzyl alcohol (2%), propylene glycol (1%), and tryptophan (1%)

TABLE 12
Aggregation analysis of Formulation B* at 60° C.

| | | % Higher | | | | | |
|---|---|---|---|---|---|---|---|
| | | Order | | | | | |
| Observed | Time | Species | % | Osmolarity | Viscosity | Syringe- | |
| pH | Point | (Aggregation) | Monomer | (mOsm/Kg) | (cp) | ability | Appearance |
| 6.0 | t₀ | 2 | 98 | 330 | 3.4 | Smooth | Clear |
| 6.1 | t₇ day | 3 | 97 | 333 | 3.6 | Smooth | Orange/ |
| | | | | | | | yellow |
| 6.0 | t₁₄ day | 5 | 95 | 350 | 4.0 | Smooth | Light brown |
| 6.5 | t₀ | 1 | 99 | 326 | 3.2 | Smooth | Clear |
| 6.4 | t₇ day | 3 | 97 | 340 | 3.6 | Smooth | Orange/ |
| | | | | | | | yellow |
| 6.3 | t₁₄ day | 9 | 91 | 344 | 3.8 | Smooth | Brown |
| 7.0 | t₀ | 1 | 99 | 325 | 3.3 | Smooth | Clear |
| 6.6 | t₇ day | 2 | 98 | 332 | 3.6 | Smooth | Orange/ |
| | | | | | | | yellow |
| 6.5 | t₁₄ day | 3 | 97 | 339 | 3.7 | Smooth | Dark brown |

*citrate buffer (20 mM), benzyl alcohol (2%), mannitol (3%), and tryptophan (1%)

TABLE 13
Aggregation analysis of Formulation C* at 60° C.

| | | % Higher | | | | | |
|---|---|---|---|---|---|---|---|
| | | Order | | | | | |
| Observed | Time | Species | % | Osmolarity | Viscosity | Syringe- | |
| pH | Point | (Aggregation) | Monomer | (mOsm/Kg) | (cp) | ability | Appearance |
| 6.0 | t₀ | 3 | 97 | 358 | 3.3 | Smooth | Clear |
| 6.1 | t₇ day | 4 | 96 | 359 | 3.4 | Smooth | Clear |
| 6.0 | t₁₄ day | 5 | 95 | 374 | 3.6 | Smooth | Yellow tint |
| 6.5 | t₀ | 1 | 99 | 353 | 3.2 | Smooth | Clear |
| 6.5 | t₇ day | 2 | 98 | 358 | 3.3 | Smooth | Clear |
| 6.5 | t₁₄ day | 2 | 98 | 352 | 3.4 | Smooth | Yellow tint |
| 7.0 | t₀ | 1 | 99 | 350 | 3.3 | Smooth | Clear |
| 6.9 | t₇ day | 1 | 99 | 354 | 3.3 | Smooth | Clear |
| 6.8 | t₁₄ day | 1 | 99 | 354 | 3.3 | Smooth | Yellow tint |

*histidine buffer (20 mM), benzyl alcohol (2%), mannitol (5%)

TABLE 14
Aggregation analysis of Formulation D* at 60° C.

| | | % Higher | | | | | |
|---|---|---|---|---|---|---|---|
| | | Order | | | | | |
| Observed | Time | Species | % | Osmolarity | Viscosity | Syringe- | |
| pH | Point | (Aggregation) | Monomer | (mOsm/Kg) | (cp) | ability | Appearance |
| 6.0 | t₀ | 2 | 98 | 328 | 2.9 | Smooth | Clear |
| 6.1 | t₇ day | 2 | 98 | 340 | 2.9 | Smooth | Clear |
| 6.4 | t₁₄ day | 3 | 97 | 298 | 3.0 | Smooth | Clear |
| 6.5 | t₀ | 1 | 99 | 323 | 3.0 | Smooth | Clear |
| 6.6 | t₇ day | 1 | 99 | 343 | 3.1 | Smooth | Clear |
| 6.5 | t₁₄ day | 2 | 98 | 352 | 3.4 | Smooth | Clear |
| 7.0 | t₀ | 1 | 99 | 320 | 3.0 | Smooth | Clear |
| 7.0 | t₇ day | 1 | 99 | 331 | 3.0 | Smooth | Clear |
| 6.9 | t₁₄ day | 1 | 99 | 358 | 3.2 | Smooth | Clear |

*histidine buffer (20 mM), benzyl alcohol (2%), propylene glycol (2%)

TABLE 15
Purity and recovery analysis of Formulation A* at 60° C.

| Observed | Time | Conjugate Conc. | | |
|---|---|---|---|---|
| pH | Point | (mg/mL) | % Recovery | % Purity |
| 6.0 | t₀ | 113.9 ± 0.7 | 100 | 82 |
| 6.0 | t_{7 day} | 98.7 ± 0.4 | 87 | 74 |
| 6.0 | t_{14 day} | 86.9 ± 0.7 | 76 | 64 |
| 6.5 | t₀ | 114.4 ± 0.8 | 100 | 82 |
| 6.4 | t_{7 day} | 99.2 ± 0.5 | 87 | 73 |
| 6.4 | t_{14 day} | 87.0 ± 0.0 | 76 | 63 |
| 7.0 | t₀ | 112.3 ± 2.8 | 100 | 82 |
| 6.6 | t_{7 day} | 93.3 ± 0.1 | 83 | 70 |
| 6.6 | t_{14 day} | 84.4 ± 0.2 | 75 | 61 |

*citrate buffer (20 mM), benzyl alcohol (2%), propylene glycol (1%), and tryptophan (1%)

TABLE 16
Purity and recovery analysis of Formulation B* at 60° C.

| Observed | Time | Conjugate Conc. | | |
|---|---|---|---|---|
| pH | Point | (mg/mL) | % Recovery | % Purity |
| 6.0 | t₀ | 112.9 ± 1.5 | 100 | 82 |
| 6.1 | t_{7 day} | 105.4 ± 1.1 | 93 | 77 |
| 6.0 | t_{14 day} | 100.7 ± 0.1 | 89 | 69 |
| 6.5 | t₀ | 111.2 ± 1.6 | 100 | 81 |
| 6.4 | t_{7 day} | 104.1 ± 0.3 | 94 | 76 |
| 6.3 | t_{14 day} | 98.8 ± 0.1 | 89 | 69 |
| 7.0 | t₀ | 113.9 ± 1.5 | 100 | 82 |
| 6.6 | t_{7 day} | 101.1 ± 0.6 | 89 | 74 |
| 6.5 | t_{14 day} | 90.6 ± 0.1 | 80 | 65 |

*citrate buffer (20 mM), benzyl alcohol (2%), mannitol (3%), and tryptophan (1%)

TABLE 17
Purity and recovery analysis of Formulation C* at 60° C.

| Observed | Time | Conjugate Conc. | | |
|---|---|---|---|---|
| pH | Point | (mg/mL) | % Recovery | % Purity |
| 6.0 | t₀ | 111.9 ± 2.1 | 100 | 82 |
| 6.1 | t_{7 day} | 104.3 ± 1.0 | 93 | 77 |
| 6.0 | t_{14 day} | 103.0 ± 1.1 | 92 | 71 |
| 6.5 | t₀ | 112.9 ± 1.1 | 100 | 82 |
| 6.5 | t_{7 day} | 107.1 ± 2.7 | 95 | 78 |
| 6.5 | t_{14 day} | 105.4 ± 0.3 | 93 | 73 |
| 7.0 | t₀ | 110.0 ± 0.0 | 100 | 81 |
| 6.9 | t_{7 day} | 107.2 ± 0.7 | 97 | 78 |
| 6.8 | t_{14 day} | 107.5 ± 0.4 | 98 | 71 |

*histidine buffer (20 mM), benzyl alcohol (2%), mannitol (5%)

TABLE 18
Purity and recovery analysis of Formulation D* at 60° C.

| Observed | Time | Conjugate Conc. | | |
|---|---|---|---|---|
| pH | Point | (mg/mL) | % Recovery | % Purity |
| 6.0 | t₀ | 111.2 ± 1.8 | 100 | 82 |
| 6.1 | t_{7 day} | 102.4 ± 0.2 | 92 | 78 |
| 6.4 | t_{14 day} | 102.7 ± 0.2 | 92 | 74 |
| 6.5 | t₀ | 109.9 ± 2.7 | 100 | 81 |
| 6.6 | t_{7 day} | 104.6 ± 0.3 | 95 | 78 |
| 6.5 | t_{14 day} | 112.4 ± 1.1 | 100 | 75 |
| 7.0 | t₀ | 109.8 ± 2.5 | 100 | 81 |
| 7.0 | t_{7 day} | 109.3 ± 1.1 | 100 | 79 |
| 6.9 | t_{14 day} | 104.1 ± 0.3 | 95 | 73 |

*histidine buffer (20 mM), benzyl alcohol (2%), propylene glycol (2%)

Of the formulations tested, the histidine/propylene glycol/benzyl alcohol and the histidine/mannitol/benzyl alcohol compositions appeared to have the best stability.

A color change was observed in all tryptophan-containing formulations after 14 days (light yellow at 25° C. and orange coloration at 60° C.) likely due to the excipient degradation. Coloration was observed to become more intense at increasing pH. A significant pH shift (approx. 0.4-0.5 pH units) was observed in the citrate/tryptophan-containing formulations at pH 7.0. The histidine/mannitol/benzyl alcohol formulations exhibited a very faint yellow discoloration, while the histidine/propylene glycol/benzyl alcohol formulations remained clear. Aggregation was observed to increase at elevated temperatures likely due to increased molecular mobility. Generally, histidine formulations showed less aggregation than citrate over time. Furthermore, histidine appeared to inhibit aggregation at elevated temperature. Polyethylene glycol formulations showed reduced aggregation at pH≥6.5 in comparison to mannitol formulations.

All formulations appeared to be chemically stable at 25° C. (purity and recovery loss≤2%). At 60° C., citrate formulations generally showed higher degradation at increasing pHs, though purity values between pH 6 and 6.5 were reported to be very similar. The citrate/mannitol/benzyl alcohol formulations showed approximately 13-17% purity loss after two weeks at 60° C., while the citrate/propylene glycol/benzyl alcohol formulations showed approximately 19-21% purity loss under similar conditions. Histidine formulations showed overall higher chemical stability than citrate formulations. The histidine/mannitol/benzyl alcohol formulations showed approximately 8-10% purity loss after two weeks at 60° C., while the histidine/propylene glycol/benzyl alcohol formulation showed 6-8% purity loss under similar conditions.

### Example 4: Filter Membrane Compatibility

Compatibility to aseptic processing was determined for the two histidine-buffered formulations from Example 4 at pH 6.5 by filtration through two membrane types: polyethersulfone (PES) and polyvinylidene fluoride (PVDF).

The following two formulations were prepared from buffer, excipient, and solid PPMO in volumetric flasks, and the pH was adjusted prior to Q.S.:
100 mg/mL antisense oligomer conjugate, 20 mM histidine, 2% propylene glycol, 2% benzyl alcohol, pH 6.5;
100 mg/mL antisense oligomer conjugate, 20 mM histidine, 5% mannitol, 2% benzyl alcohol, pH 6.5.
   Eight milliliters of the formulations were passed through the following filter membranes:
0.22 μm High Flow PES (Partorius, PN #16532, 28 mm diameter, 6.2 cm² filtration area, 100-150 μL hold-up volume;
0.22μ PVDF Membrane (EMD, PN #SLGVM33RS, 33 mm diameter, 4.5 cm² filtration area, <100 μL hold-up volume.
   The first-, fourth-, and eighth-mL filtrate were collected and tested via SCX-HPLC. Results of the study are shown in Table 19.

TABLE 19
Purity and recovery analysis of filtered samples

| | Membrane | | Conjugate Conc. | % | % | Impurity (%) | | |
|---|---|---|---|---|---|---|---|---|
| Excipients | Type | Sample | (mg/mL) | Recovery | Purity | A | B | C |
| Citrate | — | Pre- | 102.6 ± 0.7 | 100 | 78 | 3 | 10 | 9 |
| (20 mM) | | filtration | | | | | | |
| + | PES | 1 mL | 102.9 ± 0.2 | 100 | 80 | 2 | 10 | 9 |
| Benzyl alcohol | | 4 mL | 102.3 ± 0.3 | 100 | 80 | 2 | 10 | 9 |
| (2%) | | 8 mL | 100.9 ± 0.3 | 98 | 79 | 2 | 10 | 9 |
| + | PVDF | 1 mL | 103.0 ± 0.1 | 100 | 79 | 2 | 10 | 9 |
| Propylene glycol | | 4 mL | 101.8 ± 0.8 | 99 | 80 | 2 | 10 | 9 |
| (2%) | | 8 mL | 101.2 ± 0.2 | 99 | 80 | 2 | 10 | 9 |
| Citrate | — | Pre- | 101.1 ± 1.0 | 100 | 79 | 2 | 10 | 9 |
| (20 mM) | | filtration | | | | | | |
| + | PES | 1 mL | 102.6 ± 0.7 | 102 | 79 | 2 | 10 | 9 |
| Benzyl alcohol | | 4 mL | 102.3 ± 0.2 | 101 | 78 | 3 | 11 | 9 |
| (2%) | | 8 mL | 101.2 ± 1.0 | 100 | 79 | 2 | 10 | 9 |
| + | PVDF | 1 mL | 101.8 ± 0.4 | 101 | 79 | 2 | 10 | 9 |
| Mannitol | | 4 mL | 102.1 ± 0.6 | 101 | 79 | 3 | 10 | 9 |
| (5%) | | 8 mL | 102.0 ± 0.5 | 101 | 79 | 2 | 10 | 9 |

All fractions appeared clear and colorless following filtration. No impurities were introduced, and no chemical degradation was reported upon filtration with PES or PVDF membranes. Thus, both formulations are believed to be compatible with both PES and PVDF membranes.

### Example 5: Stability Under Stress Conditions

The stability of the formulation containing the antisense oligomer conjugate of formula (IVb) (100 mg/mL), histidine (20 mM), benzyl alcohol (2%), and propylene glycol (2.2%) at pH 6.5 was assessed upon exposure to photolytic stress (UV and visible light), shear force, and freeze/thaw stress.

### Photolytic Stress

The formulation was exposed to two different photolytic conditions to provide an overall exposure of visible light ranging from 1.2-3.6 k lux h (1×, 2×, and 3×ICH guidelines) and ultraviolet light ranging from 200-600 W h/m₂ (1×, 2×, and 3×ICH guidelines). Samples protected from irradiation, but otherwise exposed to the same conditions (dark samples) were also used as a control condition. Results of the study are shown in Tables 20 and 21.

TABLE 20
Results from photolytic stability testing (UV light)

| | Conjugate Conc. | | | Impurity (%) | | |
|---|---|---|---|---|---|---|
| Sample | (mg/mL) | % Recovery | % Purity | A | B | C |
| t₀ | 102.5 ± 1.1 | 100 | 79 | 2 | 11 | 9 |
| 200 W h/m2 (1X ICH) | 100.8 ± 0.3 | 98 | 79 | 1 | 10 | 9 |
| 400 W h/m2 (2X ICH) | 101.9 ± 0.5 | 100 | 79 | 1 | 11 | 9 |
| 600 W h/m2 (3X ICH) | 102.7 ± 0.2 | 100 | 79 | 1 | 10 | 9 |
| 200 W h/m2 (1X ICH) | 102.3 ± 0.5 | 100 | 79 | 2 | 10 | 9 |
| 400 W h/m2 (2X ICH) | 102.3 ± 0.3 | 100 | 79 | 2 | 10 | 9 |
| 600 W h/m2 (3X ICH) | 104.6 ± 0.3 | 102 | 80 | 1 | 10 | 9 |

TABLE 21
Results from photolytic stability testing (visible light)

| | Conjugate Conc. | | | Impurity (%) | | |
|---|---|---|---|---|---|---|
| Sample | (mg/mL) | % Recovery | % Purity | A | B | C |
| t₀ | 102.5 ± 1.1 | 100 | 79 | 2 | 11 | 9 |
| 1.2 k lux h (1X ICH) | 104.4 ± 0.2 | 102 | 79 | 1 | 10 | 9 |
| 2.4 k lux h (2X ICH) | 102.8 ± 0.2 | 100 | 79 | 1 | 11 | 9 |
| 3.6 k lux h (3X ICH) | 99.6 ± 1.4 | 97 | 78 | 2 | 11 | 10 |
| 1.2 k lux h (1X ICH) | 104.9 ± 0.3 | 102 | 80 | 1 | 10 | 8 |
| 2.4 k lux h (2X ICH) | 103.8 ± 0.7 | 101 | 80 | 1 | 10 | 8 |
| 3.6 k lux h (3X ICH) | 104.3 ± 0.4 | 102 | 80 | 1 | 10 | 8 |

No significant changes in appearance, pH, recovery, and purity were observed for UV photolytic stress the test conditions. No degradants were observed after UV exposure.

No significant purity loss or pH shift was reported after visible light exposure. No degradants were observed after visible light exposure.

### Shear Stress

The formulation was exposed to shear stress by stirring the solution in a 2-mL serum glass vial using a magnetic stirrer at 990 rpm for 48 hours. A control condition (incubation at room temperature without stirring for 48 hours) was also adopted. Results of the study are shown in Table 22.

TABLE 22
Results from shear stress stability testing

| | Conjugate Conc. | | | Impurity (%) | | |
|---|---|---|---|---|---|---|
| Sample | (mg/mL) | % Recovery | % Purity | A | B | C |
| t₀ | 102.5 ± 1.1 | 100 | 79 | 2 | 11 | 9 |
| t₂₄ₕ | 101.8 ± 0.4 | 99 | 79 | 2 | 10 | 9 |
| t₄₈ₕ | 105.4 ± 1.0 | 103 | 79 | 1 | 11 | 9 |
| t₄₈ₕ (control) | 102.5 ± 0.5 | 100 | 79 | 1 | 10 | 9 |

All samples appeared clear and colorless over the course of the study. No significant recovery loss was reported. pH values appeared to remain constant over the course of the studies. No degradants were reported throughout the study.

### Freeze-Thaw Stress

The freeze-thaw stability of the formulation was assessed. The sample was held in −80° C. for 2 hours and then at ambient temperature for 1.5 hours. The procedure was repeated for six freeze-thaw cycles. Results of the study are shown in Table 23.

TABLE 23
Results from freeze-thaw stability testing

| | Conjugate | | | | | |
|---|---|---|---|---|---|---|
| Sample | Conc. | % | % | Impurity (%) | | |
| | (mg/mL) | Recovery | Purity | A | B | C |
| t₀ | 106.4 ± | 100 | 80 | 1 | 10 | 9 |
| | 0.6 | | | | | |
| 1X | 105.3 ± | 99 | 80 | 1 | 10 | 9 |
| | 0.5 | | | | | |
| 2X | 104.8 ± | 98 | 80 | 1 | 10 | 9 |
| | 0.6 | | | | | |
| 3X | 104.3 ± | 98 | 80 | 1 | 10 | 8 |
| | 0.4 | | | | | |
| 4X | 105.2 ± | 99 | 80 | 1 | 10 | 8 |
| | 0.4 | | | | | |
| 5X | 104.9 ± | 99 | 80 | 2 | 10 | 8 |
| | 0.5 | | | | | |
| 6X | 106.9 ± | 100 | 80 | 1 | 10 | 8 |
| | 0.5 | | | | | |

All samples appeared clear and colorless up to six freeze-thaw cycles. No significant recovery or purity loss was reported. pH values appeared to remain constant over the course of the studies. A 3-fold increase in particle count was observed for the small size particles (>2 μm, <10 μm) after six freeze thaw cycles though no increase in aggregation was observed throughout the studies.

### Example 6: Shelf Life Determination

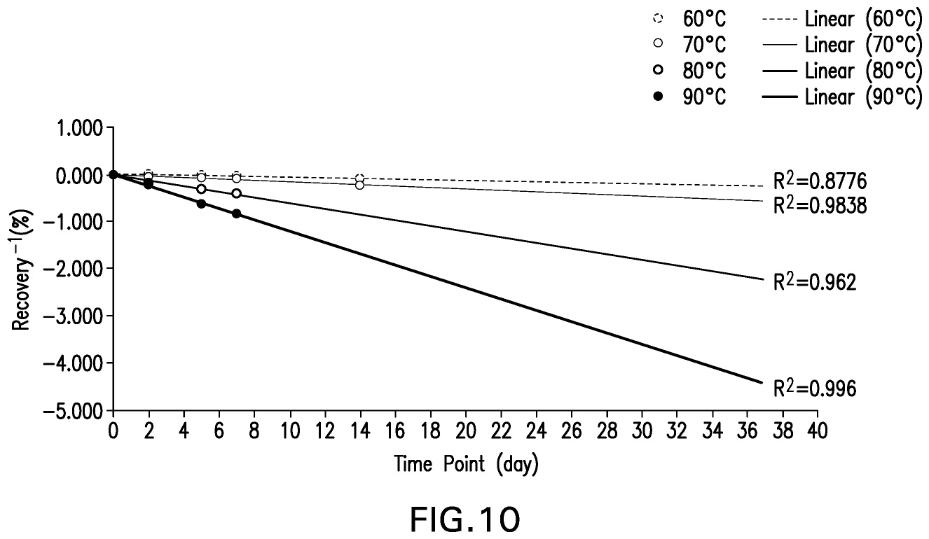

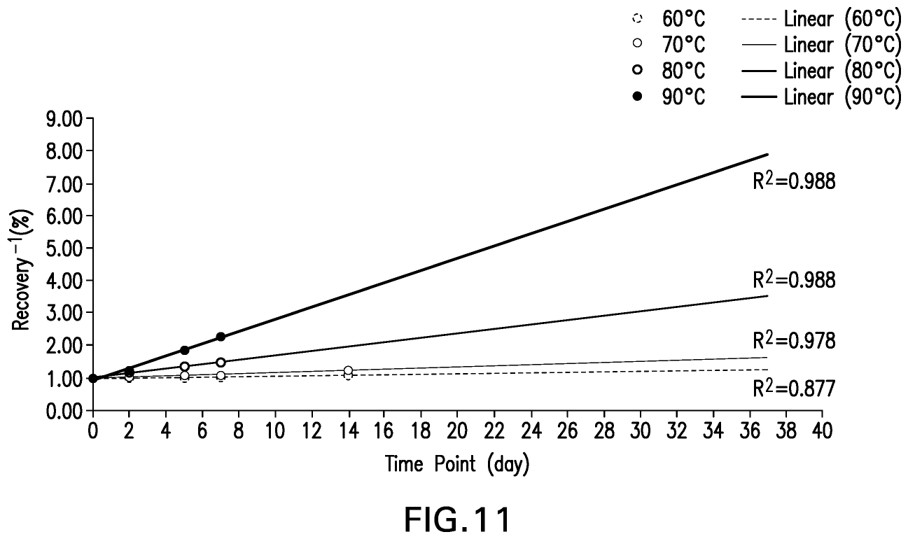

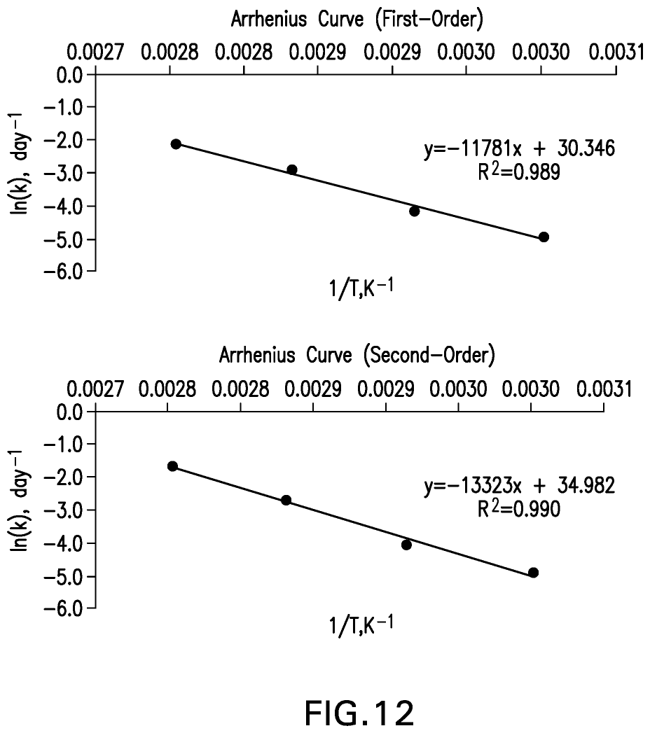

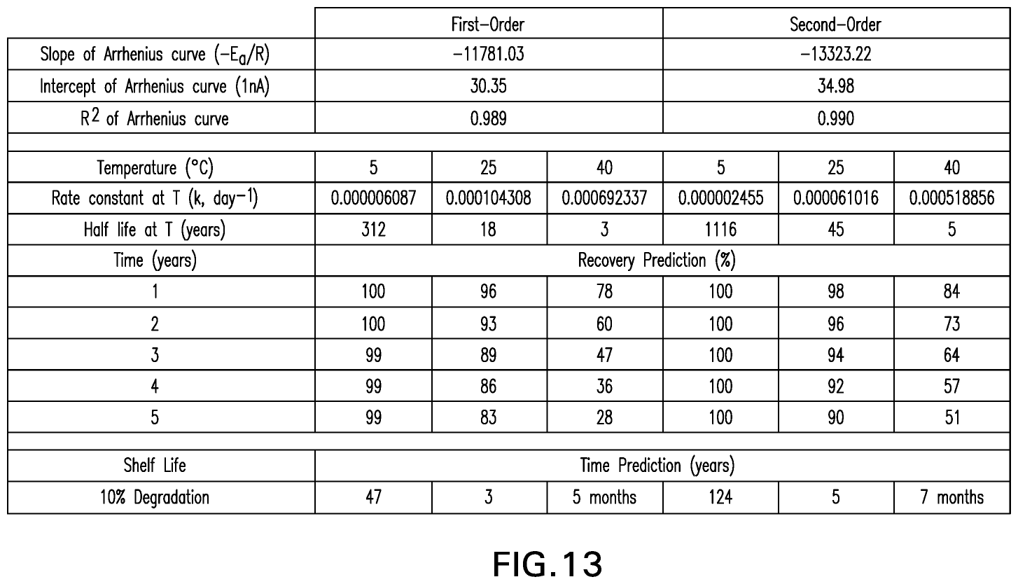

Arrhenius plots were generated by placing the formulation of Example 5 under different temperature conditions for up to two weeks. The resulting recovery values were used to calculate degradation rates, necessary to generate Arrhenius plots. The shelf life at different storage temperatures was then calculated to suggest appropriate storage conditions. Data from the degradation experiments is presented in Table 24. First and second order degradation plots are shown in FIGS. 10 and 11. Arrhenius plots are shown in FIG. 12. Estimated shelf life calculations are shown in FIG. 13.

TABLE 24
Results from freeze-thaw stability testing

| | Time | Conjugate | | | Impurity (%) | | |
|---|---|---|---|---|---|---|---|
| Condition | (days) | Conc. (mg/mL) | % Recovery | % Purity | A | B | C |
| t₀ | 0 | 102.5 ± 1.1 | 100 | 79 | 2 | 11 | 9 |
| 60° C. | 2 | 103.9 ± 0.4 | 101 | 78 | 2 | 11 | 9 |
| | 5 | 102.3 ± 0.4 | 100 | 77 | 4 | 10 | 9 |
| | 7 | 100.0 ± 0.4 | 98 | 76 | 4 | 11 | 9 |
| | 14 | 93.6 ± 0.4 | 91 | 68 | 10 | 13 | 10 |
| 70° C. | 2 | 98.2 ± 0.7 | 96 | 75 | 5 | 11 | 9 |
| | 5 | 95.0 ± 1.5 | 93 | 69 | 10 | 12 | 10 |
| | 7 | 93.5 ± 2.2 | 91 | 69 | 10 | 11 | 10 |
| | 14 | 81.8 ± 1.0 | 80 | 61 | 16 | 12 | 11 |
| 80° C. | 2 | 86.0 ± 0.4 | 84 | 63 | 13 | 14 | 10 |
| | 5 | 75.4 ± 0.1 | 74 | 59 | 15 | 15 | 11 |
| | 7 | 68.7 ± 2.3 | 67 | 53 | 20 | 15 | 11 |
| 90° C. | 2 | 83.2 ± 0.2 | 81 | 62 | 13 | 14 | 11 |
| | 5 | 55.0 ± 0.8 | 54 | 43 | 29 | 17 | 11 |
| | 7 | 44.8 ± 0.1 | 44 | 32 | 38 | 19 | 11 |

Computational analysis suggests stability of the formulation at 2-8° C. for at least five-year storage and at 25° C. for a two-year storage. Short-term excursions to higher temperatures (e.g., 24 hours at 40° C.) appear to be permissible during formulation handling and transport.

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

Those skilled in the art will recognize, or be able to ascertain using no more than routine experimentation, many equivalents of the specific embodiments of the disclosure described herein. Such equivalents are intended to be encompassed by the following claims.

1. A pharmaceutical composition comprising benzyl alcohol and an antisense oligomer conjugate of formula (I): 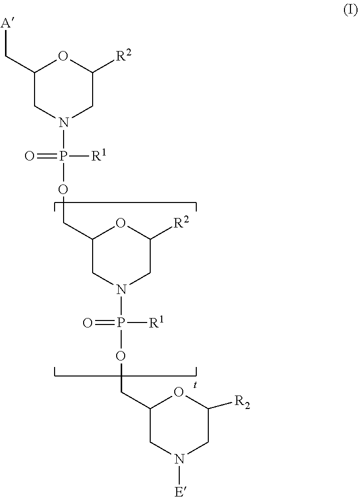
or a pharmaceutically acceptable salt thereof,
wherein:
A′ is selected from —OH, 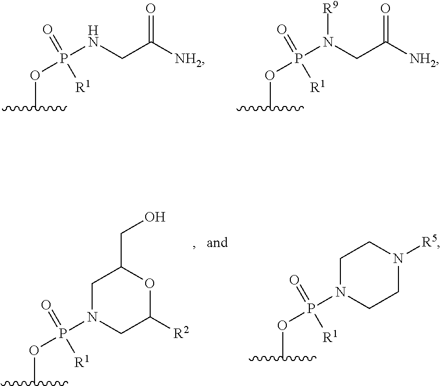
wherein
R⁵ is —C(O)(O-alkyl)ₓ-OH, wherein x is 3-10 and each alkyl group is, independently at each occurrence, C₂₋₆-alkyl,
or R⁵ is selected from —H, —C(O)C₁₋₆-alkyl, trityl, monomethoxytrityl, —(C₁₋₆-alkyl)-R⁶, —(C₁₋₆-heteroalkyl)-R⁶, —C₆₋₁₀-aryl-R⁶, 5- to 10-membered heteroaryl-R⁶, —C(O)O—(C₁₋₆-alkyl)-R⁶, —C(O)O—(C₆₋₁₀-aryl)-R⁶, —C(O)O-(5- to 10-membered heteroaryl)-R⁶, and 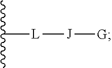
R⁶ is selected from —OH, —SH, and —NH₂, or R⁶ is O, S, or NH, each of which is covalently linked to a solid support;
R⁹ is C₁₋₆-alkyl;
each R¹ is independently selected from —OH and —N(R³)(R⁴), wherein each R³ and R⁴ is, independently at each occurrence, —H or —C₁₋₆-alkyl;
each R² is independently, at each occurrence, selected from —H, a nucleobase, and a nucleobase functionalized with a chemical protecting group, wherein the nucleobase and the nucleobase functionalized with a chemical protecting group, independently at each occurrence, comprise a ring selected from pyridine, pyrimidine, purine, and deaza-purine;
t is 8-40;
E′ is selected from —H, —C₁₋₆-alkyl, —C(O)C₁₋₆-alkyl, benzoyl, stearoyl, trityl, monomethoxytrityl, dimethoxytrityl, trimethoxytrityl, 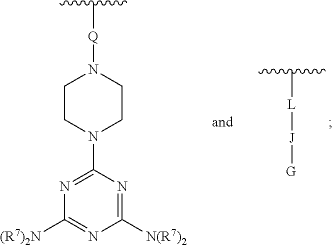
wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)₆NHC(═NH)NH₂;
L is a linking amino acid, wherein L is covalently linked by an amide bond to the N-terminus or C-terminus of J;
J is a cell-penetrating peptide;
G is selected from —H, —C(O)C₁₋₆-alkyl, benzoyl, and stearoyl, wherein G is covalently linked to J; and
wherein at least one of the following is true:
(1) A′ is 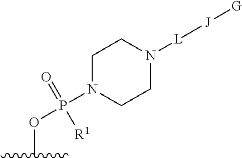
or (2) E′ is

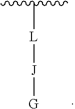

2. The pharmaceutical composition of claim 1, wherein E′ is selected from —H, —C₁₋₆-alkyl, —C(O)C₁₋₆-alkyl, benzoyl, stearoyl, trityl, monomethoxytrityl, dimethoxytrityl, trimethoxytrityl, and

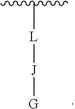

3. (canceled)

4. The pharmaceutical composition according to claim 1, wherein A′ is selected from:

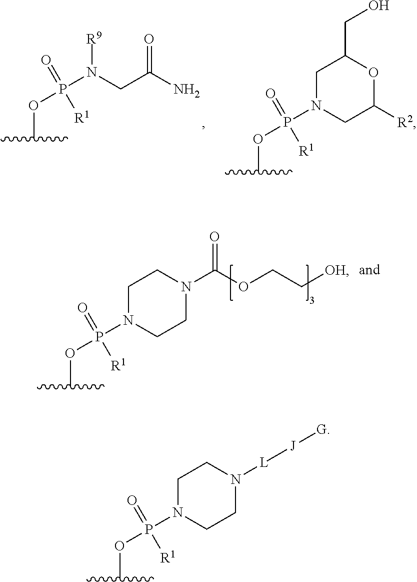

5-7. (canceled)

8. The pharmaceutical composition according to claim 1, wherein L is glycine, proline, or β-alanine.

9-11. (canceled)

12. The pharmaceutical composition according to claim 1, wherein J is selected from SEQ ID NOS: 5-21.

13. The pharmaceutical composition according to claim 1, wherein G is selected from —H, —C(O)CH₃, benzoyl, and stearoyl.

14-16. (canceled)

17. The pharmaceutical composition according to any ene of claim 1, wherein each R² is a nucleobase, and all R² groups taken together form a targeting sequence; wherein the targeting sequence is selected from:
SEQ ID NO: 3
(CTGAGCCGCTGGCAGATGCCTTGTC) wherein t is 23;
and
SEQ ID NO: 4
(GAGGAGATGGGTCCACCCACCTGGG) wherein t is 23.

18. The pharmaceutical composition according to claim 1, wherein the antisense oligomer conjugate is of formula (IA): 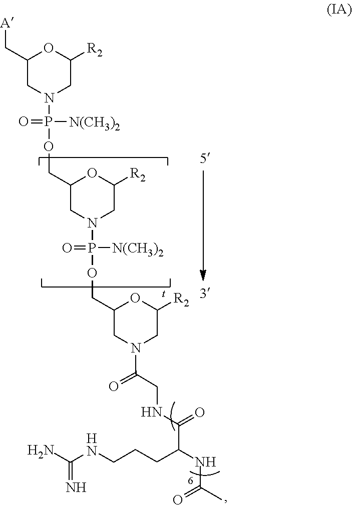
or a pharmaceutically acceptable salt thereof, wherein:
A′ is a moiety selected from:

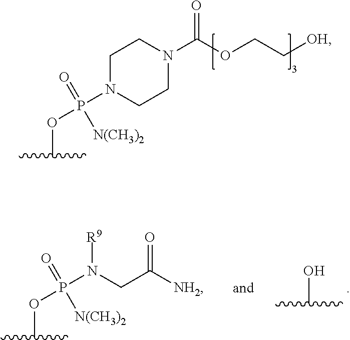

19. The pharmaceutical composition according to claim 1, wherein the antisense oligomer conjugate is of formula (II): 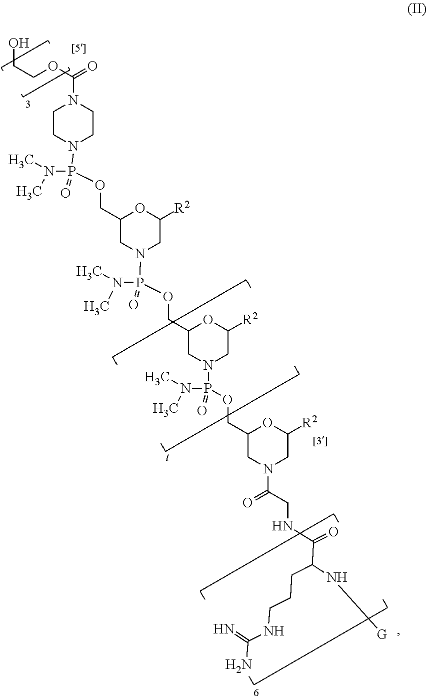
or a pharmaceutically acceptable salt thereof.

20. The pharmaceutical composition according to claim 1, wherein the antisense oligomer conjugate is an HCl salt.

21. (canceled)

22. The pharmaceutical composition according to claim 1, wherein the antisense oligomer conjugate is of Formula (IIA): 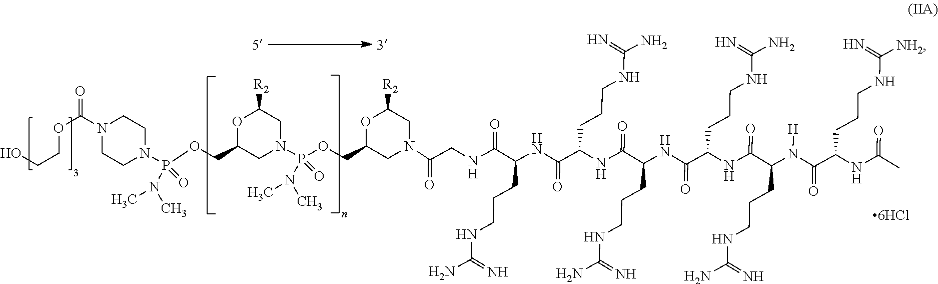
wherein n is 9-39.

23. (canceled)

24. The pharmaceutical composition according to claim 1, wherein the pharmaceutical composition further comprises one or more of histidine, citrate, mannitol, propylene glycol, glycerin, arginine, lysine, tryptophan, or phenol.

25. (canceled)

26. The pharmaceutical composition according to claim 1, wherein the pharmaceutical composition has a pH range of 6.0 to 7.0.

27. (canceled)

28. The pharmaceutical composition according to claim 1, wherein the composition comprises 2% weight by volume of benzyl alcohol and the composition has a pH of 6.5.

29. (canceled)

30. The pharmaceutical composition according to claim 1, wherein the pharmaceutical composition further comprises histidine and propylene glycol.

31. The pharmaceutical composition according to claim 30, wherein the composition comprises 2% weight by volume of benzyl alcohol, 2-3% weight by volume of propylene glycol, and the composition has a pH of 6.5.

32. (canceled)

33. The pharmaceutical composition according to claim 1, wherein the pharmaceutical composition further comprises histidine and mannitol.

34. The pharmaceutical composition according to claim 33, wherein the composition comprises 2% weight by volume of benzyl alcohol, 5% weight by volume of mannitol, and the composition has a pH of 6.5.

35. (canceled)

36. The pharmaceutical composition according to claim 22, wherein the targeting sequence is selected from:
SEQ ID NO: 3
(CTGAGCCGCTGGCAGATGCCTTGTC),
and
n is 24;
and
SEQ ID NO: 4
(GAGGAGATGGGTCCACCCACCTGGG),
and
n is 24.

37-40. (canceled)

41. A method for treating Hutchinson-Gilford progeria syndrome (HGPS) in a subject in need thereof comprising administering to the subject the pharmaceutical composition comprising benzyl alcohol and an antisense oligomer conjugate of formula (I); 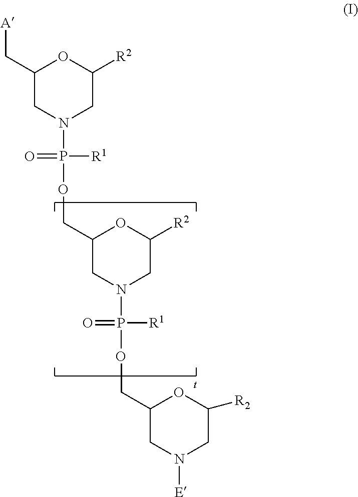
or a pharmaceutically acceptable salt thereof,
wherein:
A′ is selected from —OH, 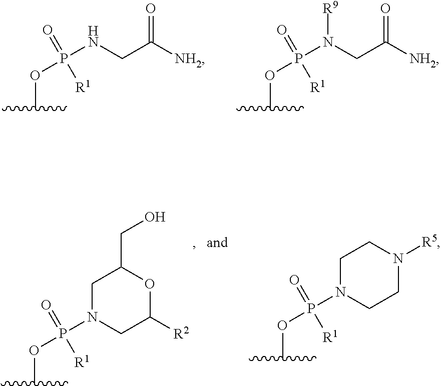
wherein
R⁵ is —C(O)(O-alkyl)ₓ-OH, wherein x is 3-10 and each alkyl group is, independently at each occurrence, C₂₋₆-alkyl,
or R⁵ is selected from —H, —C(O)C₁₋₆-alkyl, trityl, monomethoxytrityl, —(C₁₋₆-alkyl)-R⁶, —(C₁₋₆-heteroalkyl)-R⁶, —C₆₋₁₀-aryl-R⁶, 5- to 10-membered heteroaryl-R⁶, —C(O)O—(C₁₋₆-alkyl)-R⁶, —C(O)O—(C₆₋₁₀-aryl)-R⁶, —C(O)O-(5- to 10-membered heteroaryl)-R⁶, and 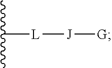
R⁶ is selected from —OH, —SH, and —NH₂, or R⁶ is O, S, or NH, each of which is covalently linked to a solid support;
R⁹ is C₁₋₆-alkyl;
each R¹ is independently selected from —OH and —N(R³)(R⁴), wherein each R³ and R⁴ is, independently at each occurrence, —H or —C₁₋₆-alkyl:
each R² is independently, at each occurrence, selected from —H, a nucleobase, and a nucleobase functionalized with a chemical protecting group, wherein the nucleobase and the nucleobase functionalized with a chemical protecting group, independently at each occurrence, comprise a ring selected from pyridine, pyrimidine, purine, and deaza-purine;
t is 8-40;
E′ is selected from —H, —C₁₋₆-alkyl, —C(O)C₁₋₆-alkyl, benzoyl, stearoyl, trityl, monomethoxytrityl, dimethoxytrityl, trimethoxytrityl, 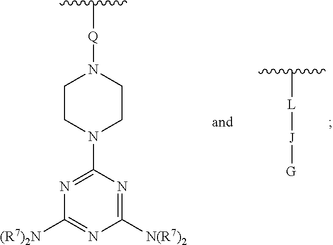
wherein
Q is —C(O)(CH₂)₆C(O)— or —C(O)(CH₂)₂S₂(CH₂)₂C(O)—;
R⁷ is —(CH₂)₂OC(O)N(R⁸)₂, wherein R⁸ is —(CH₂)(NHC(═NH)NH₂;
L is a linking amino acid, wherein L is covalently linked by an amide bond to the N-terminus or C-terminus of J;
J is a cell-penetrating peptide;
G is selected from —H, —C(O)C₁₋₆-alkyl, benzoyl, and stearoyl, wherein G is covalently linked to J; and
wherein at least one of the following is true;
(1) A′ is 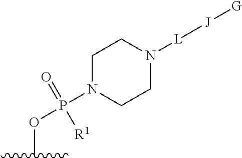
or (2) E′ is [chemical structure]
